(19) United States
(12) Patent Application Publication
(10) Pub. No.: US 2006/0015188 A1
(43) Pub. Date: Jan. 19, 2006

(54) Prosthesis and method of implantation

(75) Inventor: James B. Grimes, Bakersfield, CA (US)

Correspondence Address: SENNIGER POWERS, ONE METROPOLITAN SQUARE, 16TH FLOOR, ST LOUIS, MO, 63102, US

(73) Assignee: Nexus Consulting Limited

(21) Appl. No.: 10/988,458

(22) Filed: Nov. 12, 2004

Related U.S. Application Data
(60) Provisional application No. 60/589,173, filed on Jul. 17, 2004.

(51) Int. Cl. A61F 2/36; A61F 2/30; A61B 17/90

(52) U.S. Cl. 623/23.19; 623/23.22; 623/23.31; 623/23.44; 623/23.48; 606/96

(57) ABSTRACT

A bone prosthesis for implantation at a joint is adapted to closely replicate the normal loading of the femur and is suitable for implantation using a single incision anterior approach, a form of minimally invasive surgery (MIS). The bone prosthesis comprises a stem adapted for orientation with a medial trabecular stream of the femur.

### CROSS REFERENCE TO RELATED APPLICATION

This application claims priority from Provisional Application Ser. No. 60/589,173 filed on Jul. 17, 2004, which is hereby incorporated by reference in its entirety.

### BACKGROUND

The present invention relates to an implantable prosthesis for implantation in a bone such as a femur.

A femoral head-neck prosthesis that fails to replicate normal loading conditions will change the stress distribution through the femur. As mentioned in U.S. Pat. No. 4,998,937, according to Wolff's law these changes in stress distribution eventually cause alterations in the internal structure of the bone. Those portions subject to a lesser stress than before are likely to deteriorate and those subject to greater stress than before are likely to thicken. But if the stress is too great and applied over an extended period, bone cells may be killed.

As shown in FIG. 1, the human femur F has two externally visible axes: the axis of the femoral neck AX-1 and the axis of the femoral shaft AX-2. However, the bone is not loaded along either of these two visible axes, but rather is loaded through a third axis (parallel to the average compression loading vector), which is not externally apparent. In response to compressive loading and the strain energy density experienced by the femur F, reinforcing lines of bone, which are called compression trabeculae, form within the femur. The collection of these reinforcing lines is the compression trabecular stream or medial trabecular stream MTS. The particular collection of trabeculae in the femur neck N, as shown in FIG. 1, is referred to as the medial trabecular stream MTS, and the average direction of the medial trabecular stream may be referred to as the medial trabecular stream axis AX-3. Angle θ, which axis AX-3 makes with the central longitudinal axis of the femoral shaft AX-2, generally ranges from 138 to 171 degrees. In practice, this angle may be measured from a profile X-ray of the hip between the axis AX-3 and a lateral surface of the femur F. The use of the medial trabecular stream MTS to position a femoral prosthesis is discussed in U.S. Pat. No. 4,998,937 (hereinafter '937), U.S. Pat. No. 6,740,120 ('120) and U.S. Pat. No. 6,273,915 ('915), all of which are incorporated herein by reference.

In most cases, the stem cannot be aligned exactly with the MTS axis for anatomical reasons, e.g., because the axis extends through the medial neck cortex above the lesser trochanter. Previous transosseous implants, such as the implants shown in the '915 and '120 patents, were installed in “offset” alignment so that the stem was aligned with axis AX-4, parallel to the MTS axis, e.g., offset from the axis so that the bore did not extend through the neck cortex. A disadvantage of “offset” alignment is that it creates a bending moment on the stem that may excessively strain parts of the femur, e.g., the femoral neck. Accordingly, other types of alignments may be desired in some femoral implant applications.

Additionally, previous stems have included longitudinal splines for preventing rotation of the stem. The splines are evenly spaced around the circumference of the stem. While the splines prevent rotation, they have the disadvantage of tending to cause the stem to deviate horizontally during implantation (known in the art as “going into varus”) as the implant is impacted or driven into the bore. Thus, an improved spline configuration is desired.

Also, femoral transosseous prostheses typically require at least two incisions. However, it would be desirable to minimize the number of incisions to reduce recovery time and the risk of infection.

### SUMMARY OF THE INVENTION

One aspect of the invention is directed to a bone prosthesis for implantation at a joint. The prosthesis comprises a stem sized and shaped for implantation in a bone at the joint, the stem having a proximal portion, a distal portion and a longitudinal axis extending therethrough. The distal portion has an outer periphery including splined sections of longitudinally extending splines and non-splined sections separating the splined sections. The splined sections and non-splined sections being constructed and arranged for facilitating implantation and for inhibiting cracking of the bone.

In another aspect, the prosthesis comprises a stem adapted for implantation through a bore formed in a bone at the joint, the bore having an entrance at one side of the bone and an exit at an opposite side. The stem includes a distal portion and a distal tip at an end of the distal portion. The distal portion and distal tip are formed integrally as one piece. The distal portion has an outer periphery including splined sections of longitudinally extending splines. The tip has a smooth, curved leading edge and non-splined, smooth section disposed between the leading edge and the distal portion for facilitating insertion of the tip through the entrance and through the exit of the bore and for facilitating centering of the splines of the distal portion.

In yet another aspect, a prosthesis is adapted for transosseous implantation in a femur having a bore and an adjacent seat formed therein. The prosthesis comprises a collar, a neck mounted on one side of the collar, and a stem extending from the collar on the opposite side of the collar from the neck. The collar includes a lip for engaging the seat formed in the femur so as to inhibit withdrawal of the prosthesis from the seat and the bore while allowing compression of the prosthesis against the bone.

In still another aspect, the collar is sized and shaped for engaging the seat formed in the femur so as to inhibit withdrawal of the prosthesis from the seat and the bore while allowing compression of the prosthesis against the bone.

In an additional aspect, the prosthesis comprises a neck adapted to receive a ball thereon and having a neck longitudinal axis, a collar on which the neck is mounted and a stem extending from the collar on the opposite side of the collar from the neck. The stem includes a proximal portion adjacent the collar, a central portion and a distal portion opposite the proximal portion. The distal portion has a distal tip. The proximal and central portions being symmetric about a stem longitudinal axis. The stem longitudinal axis being angled relative to the neck longitudinal axis and forming an acute angle relative to the collar.

In another aspect, the prosthesis comprises a first assembly including a collar having a first side and a second side opposite the first side adapted to engage the femur. The first assembly also includes a neck fixed to the first side of the collar and adapted to receive a ball thereon. A second assembly includes a generally straight stem adapted for transosseous implantation in the bore. The second assembly is securable to the first assembly for extending from the second side of the collar.

In still another aspect, a method is adapted for implanting a femoral prosthesis. The femur has a shaft, a neck at the upper end of the shaft at the medial side of the femur, and a trabecular stream. The method comprises the steps of determining the axis of the trabecular stream of the femur, and forming a seat on the femoral neck. A bore is drilled along a line through the shaft of the femur co-linear with the medial trabecular stream generally at the lateral side of the femur so as to increase the bore length through the femur and to decrease the bending moment on the prosthesis. Finally, a stem of the prosthesis is inserted in the bore extending through the shaft to the lateral side of the femur so that a stem axis is co-linear with the medial trabecular stream.

In another aspect, the method comprises inserting a stem of the prosthesis in the bore and orienting the stem so that one of the non-splined sections is positioned superolaterally in the femur and another of the non-splined sections is positioned inferomedially in the femur.

In still another aspect, a prosthesis comprises a first assembly including a collar having a first side and a second side opposite the first side adapted to engage the femur, a neck mounted on a first side of the collar and adapted to receive a ball thereon, and a proximal stem secured to the second side of the collar. A second assembly includes a distal stem adapted for implantation in the bore, the second assembly being securable to the first assembly for extending from the proximal stem.

In yet another aspect, the prosthesis comprises a collar having a first side and a second side opposite the first side adapted to engage the femur. A neck is mounted on a first side of the collar and has a longitudinal neck axis. A stem is sized and shaped for implantation in a bore through the bone. The stem includes a proximal portion and a distal portion having a longitudinal distal axis generally co-linear with the neck axis and offset from the proximal portion.

In yet a further aspect, a method is adapted for implanting a femoral prosthesis in a femur. The prosthesis comprises a collar, and stem including a proximal portion, a distal portion and a cement restrictor around the proximal portion. The femur has a shaft and a neck at the upper end of the shaft at the medial side of the femur. The method comprises the steps of forming a seat on the femoral neck, and drilling a bore along a line through the shaft of the femur to extend from the neck of the femur down through the lateral side of the femur. The stem of the prosthesis is partially inserted in the bore extending through the shaft to the lateral side of the femur. Cement is placed around the proximal portion of the stem such that the restrictor inhibits the cement from flowing down toward the distal portion. In the final step, the prosthesis is impacted into the bone so that the collar contacts the seat.

In still another aspect, a method is adapted to incrementally adjust a location of a guide pin in forming a bore for transosseous prosthetic implantation. The method comprises withdrawing the guide pin from a first guide hole and determining a location of a final guide slot. Another step includes placing a side-cutting burr in the first guide hole and rotating the burr while forcing the burr against one edge of the hole to expand the hole and thereby form the final guide slot. A further step includes inserting the guide pin in the guide slot.

In yet another aspect, a guide for use during the implantation of a prosthesis comprises a cylindrical body having a top, a bottom, an inner wall and an outer wall. The inner wall defines an opening for allowing the shaft to pass therethrough. At least one passage extends from the top of the body to the bottom of the body for allowing fluid to pass through the guide for direct fluid contact with the reamer.

In still a further aspect, a stem for a femoral prosthesis adapted for transosseous implantation comprises a first diameter cylinder tapering to a smaller second diameter in a distal portion of the stem, and a medial side of the stem lying along a straight line.

### BRIEF DESCRIPTION OF THE DRAWINGS

FIG. 1 is a fragmentary front elevational view of an intact femur showing the medial trabecular stream (MTS) of the femur;

FIG. 2 is a fragmentary cross section of an upper femur showing a femoral prosthesis of an embodiment of the present invention implanted in the femur (the prosthesis being shown in full lines);

FIG. 3 is an elevational view of the prosthesis of FIG. 2;

FIG. 4 is a perspective view of the prosthesis;

FIG. 5A is an exploded view of the prosthesis;

FIG. 5B is an enlarged view of a stem of the prosthesis;

FIG. 5C is an enlarged side view of a distal tip of the stem;

FIG. 5D is an enlarged perspective view of the distal tip;

FIG. 6 is a bottom view of a collar-neck assembly of the prosthesis;

FIG. 7 is a section view taken along lines 7—7 of FIG. 5B;

FIG. 8 is a perspective of a collar-neck assembly of another embodiment of the invention;

FIG. 8A is a perspective of the collar-neck assembly of FIG. 8 along with an impactor;

FIG. 9 is a top view of the assembly of FIG. 8;

FIGS. 10A-10E are section views taken along lines 10-10 of FIG. 9 and showing several alternate constructions of the assembly

FIGS. 11A-11C are fragmentary cross sections similar to FIG. 2 and showing formation of a seat in the femur;

FIGS. 12A-12C are elevational views of femurs;

FIG. 13 is a fragmentary cross section of an upper femur similar to FIG. 2 but showing a prosthesis of another embodiment of the present invention;

FIGS. 14-19 show a progression of steps of a method for implanting a prosthesis of another embodiment;

FIGS. 20-21 show a progression of steps for an embodiment similar to FIGS. 14-19;

FIG. 22 is a fragmentary cross section showing yet another embodiment;

FIGS. 23-28 show a progression of steps for yet another method of the invention;

FIGS. 29A-C, 30A-B, 31A-B show variations of a stem of the invention;

FIGS. 32-33 show cemented prostheses of the invention;

FIG. 34 is a perspective a fenestrated guide having discrete passages;

FIG. 35 is fragmentary cross section showing the fenestrated guide of FIG. 34 positioned in the femur; and

FIG. 36 is a perspective of another embodiment of a fenestrated guide having connected passages.

Corresponding reference characters indicate corresponding parts throughout the several views of the drawings.

### DETAILED DESCRIPTION OF THE PREFERRED EMBODIMENTS

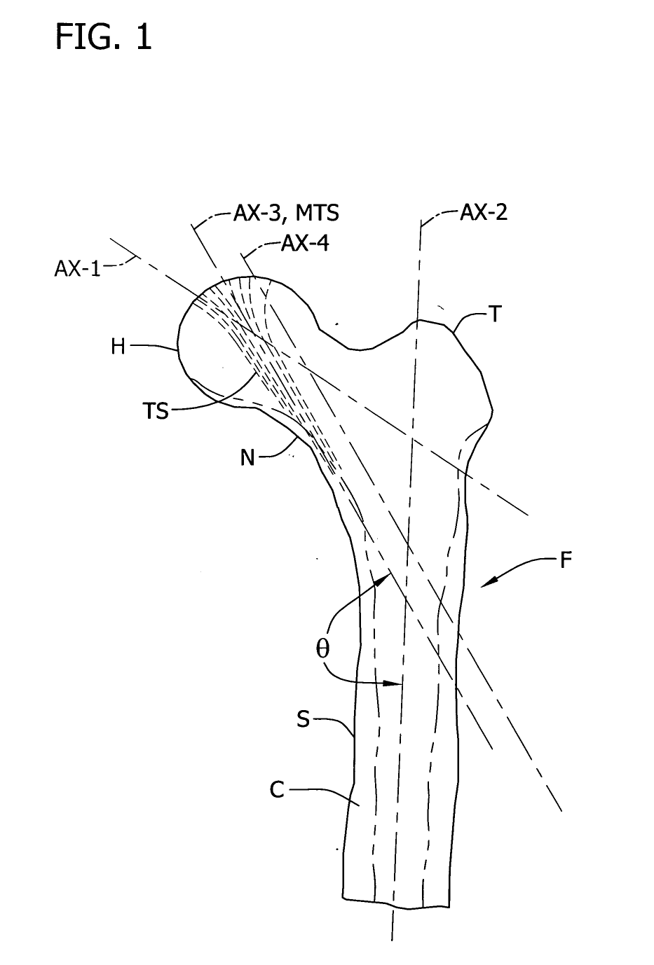

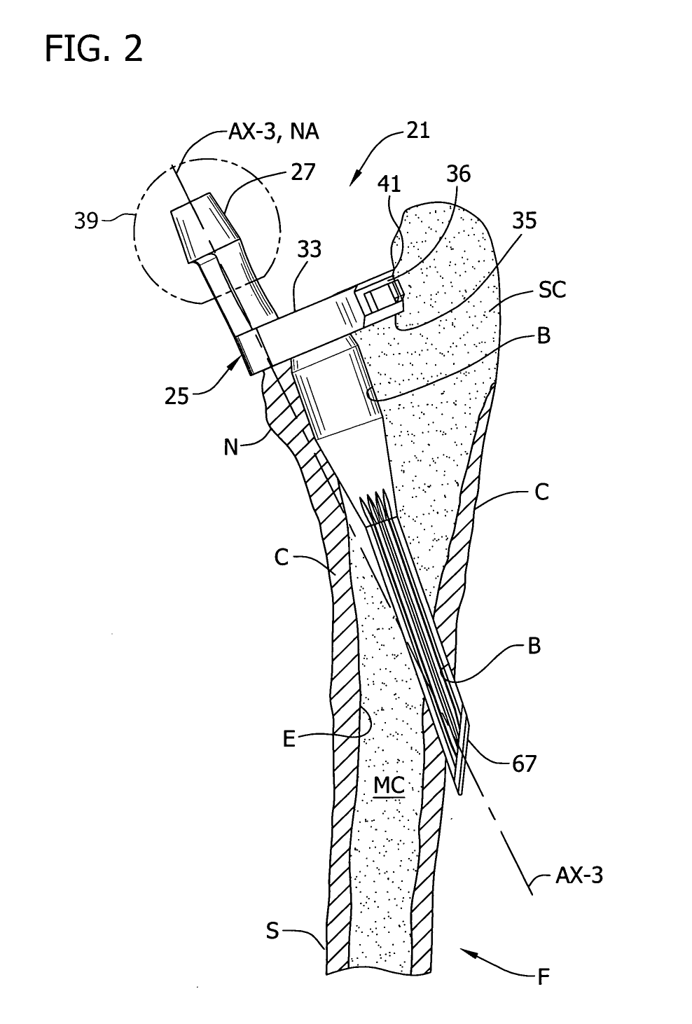

Referring now to the drawings and in particular to FIGS. 1-2, a transosseous prosthesis of an embodiment of the invention is designated in its entirety by the reference numeral 21. In this embodiment, the prosthesis is suitably sized and shaped for implantation in a femur F, though it is to be understood that the prosthesis may be sized and shaped for implantation in other bones, e.g., the humerus. The femur includes a femoral shaft S, a femoral head H (it is removed in FIG. 2), neck N, a trabecular stream TS and a greater trochanter T at the upper end of the shaft at the lateral side of the femur. The femur F has a hard layer of cortical bone C adjacent the surface of the bone, relatively soft cancellous bone SC, a medullary canal MC, and endosteum E inside the femur.

As implanted, the transosseous prosthesis 21 extends through a bore B generally from the resected femoral neck N diagonally across the medullary canal MC and out an opposite side of the femur. The prosthesis 21 will usually extend out posterolaterally (i.e., from the posterolateral side of the femur F), but might extend laterally or anterolaterally in cases of neutral version or retroversion, respectively. It should be noted that some features of the prosthesis 21 can be incorporated into non-transosseous, intramedullary prostheses. The prosthesis 21 is of the type that need not be cemented into the femur F, but is secured by mechanical interconnection of the prosthesis with the bone. The prosthesis 21 is constructed so that it is securely held in the bone from rotation (about its longitudinal axis) and toggling (perpendicular to the longitudinal axis, i.e., anterior-posterior and medial-lateral) motion, while permitting axial micromotion to achieve a natural bone loading condition thereby to preserve the bone.

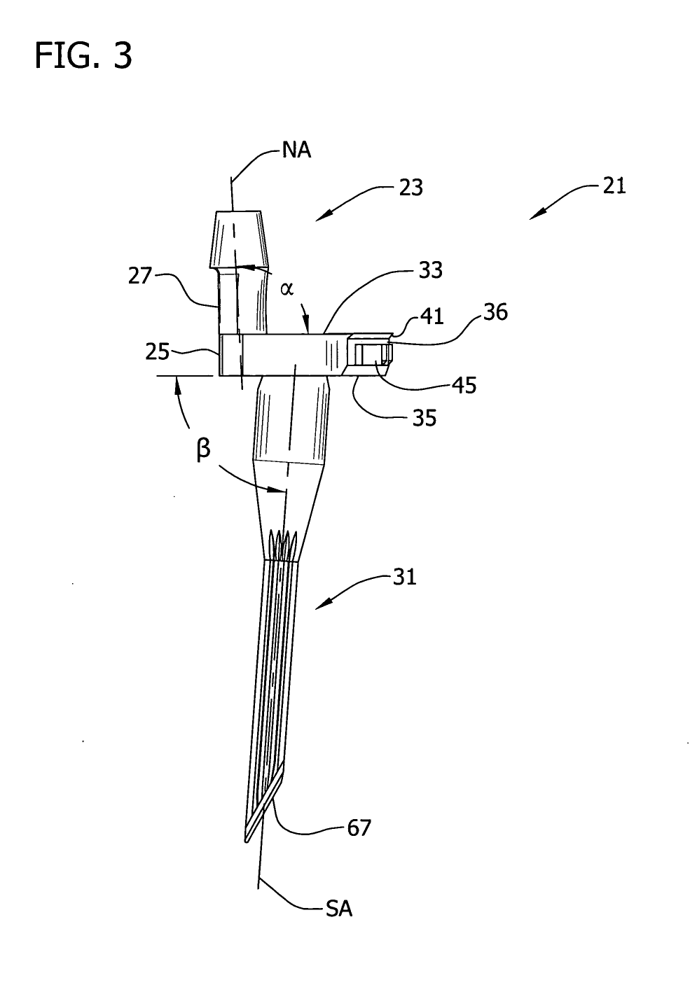

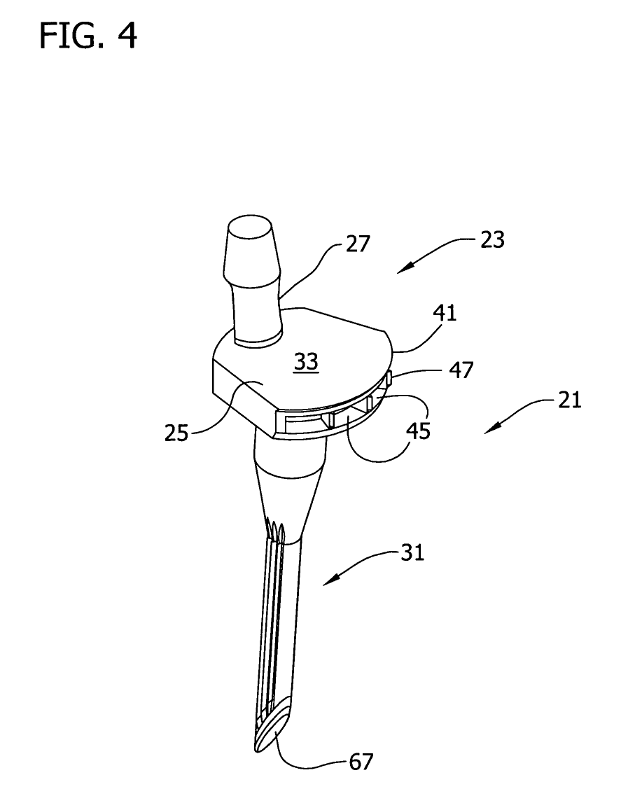

Referring to FIGS. 3-6, the prosthesis 21 of this embodiment is modular and comprises a collar-neck assembly (broadly, a first assembly) generally designated 23 that includes a collar 25 and a neck 27. The prosthesis further comprises a second assembly including a stem generally designated 31. The collar includes an upper surface 33, a lower surface 35 generally on an opposite side of the collar from the upper surface, and a lateral edge 36 extending therebetween at a lateral side of the collar. The lower surface 35 and the lateral edge are sized and shaped to engage the bone (e.g., cortical and cancellous bone of the femur F). In particular, the lower surface 35 is adapted to transfer forces (load) to the bone. As shown in FIG. 6, the lower surface 35 includes an opening 37 for receiving the stem. The opening 37 is perpendicular to the lower surface, but may alternatively be angled so that the stem extends from the collar at an angle.

The neck 27 of the collar-neck assembly 23 extends upwardly from the upper surface 33 and is adapted to receive a ball 39 (shown in phantom in FIG. 2) thereon. The neck 27 has a longitudinal axis NA that forms an angle α with the upper surface 33 of the collar 25 (FIG. 3).

In this embodiment, the neck 27 is disposed at an oblique angle α of about 94° relative to the upper surface 33 of the collar 25 (i.e., the neck is not perpendicular to the collar), but the neck may extend at an angle of between about 90° to about 100°. In conjunction with the intercept alignment discussed below, the longitudinal neck axis NA extends at an angle relative to a longitudinal stem axis SA of about 80, but the angle between the axis is suitably between about 3 and about 150. It is also contemplated that the neck axis NA and stem axis SA be parallel. The neck and stem axes may be disposed at a variety of angles relative to the collar 25. The angles are chosen to conform to the individual femur F. For example, it is contemplated within the scope of the invention for the neck 27 to extend perpendicular to the collar 25 while the stem 31 extends at an acute or oblique angle, or in the reverse, the neck extends obliquely or acutely and the stem extends perpendicular.

In this embodiment, the collar-neck assembly 23 is integrally formed as one piece. The stem 31 and collar-neck assembly can be joined by suitable joining means, such as described below. However, it is contemplated within the scope of this invention to form the collar 25, the neck 27 and the stem 31 integrally as one piece or any number of separate pieces.

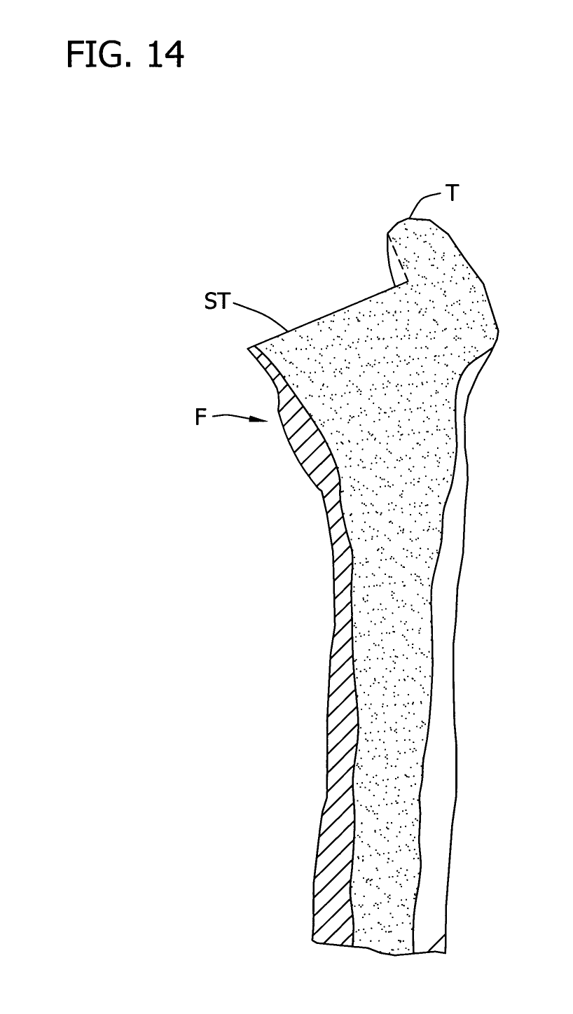

Referring to FIGS. 2-5D, compression of the collar 25 against the femoral neck N promotes full strain transmission in the proximal (upper) femur to thereby inhibit bone loss after implantation. To facilitate compression, the prosthesis 21 allows axial translation (or micromotion) of the stem 31 through the bore B. Accordingly, the stem 31 of this embodiment is typically not cemented to the femur F, and the stem is constructed to inhibit biological ingrowth into the stem. However, unrestricted compression of the collar 25 against the femoral neck N could permit the collar to distract or withdraw from its seated position under certain circumstances. The collar 25 of this embodiment includes a lip 41 for engaging a seat ST formed in the femoral neck N (see FIGS. 2 and 14) so as to inhibit distraction or withdrawal of the prosthesis 21 from the seat and the bore B while allowing compression of the prosthesis against the bone.

The lip 41 is a mechanical interlock between the collar 25 and the femur F that allows compression of the collar but also inhibits distraction. Forces that would tend to distract the prosthesis 21 from the femur F are likely to be minimal. The resistance to distraction achieved by the lateral collar lip 41 is expected to be necessary only until sufficient bone ingrowth into the collar 25 occurs. However, mechanical resistance to distraction may provide additional stability and encourage osseointegration in marginal cases.

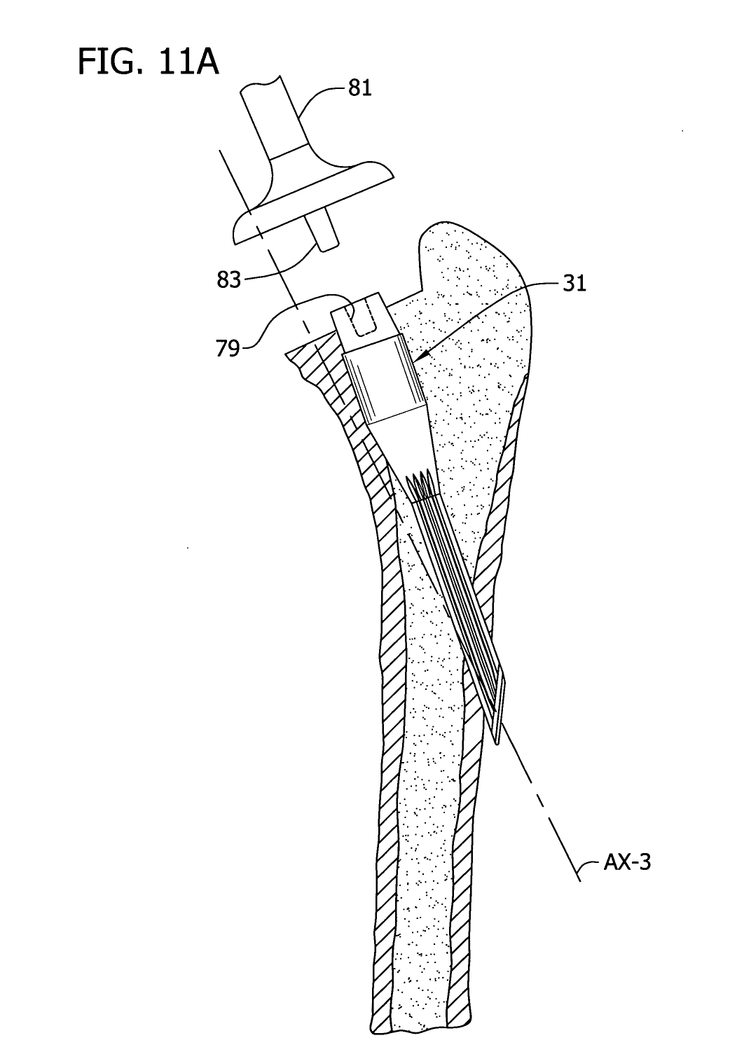

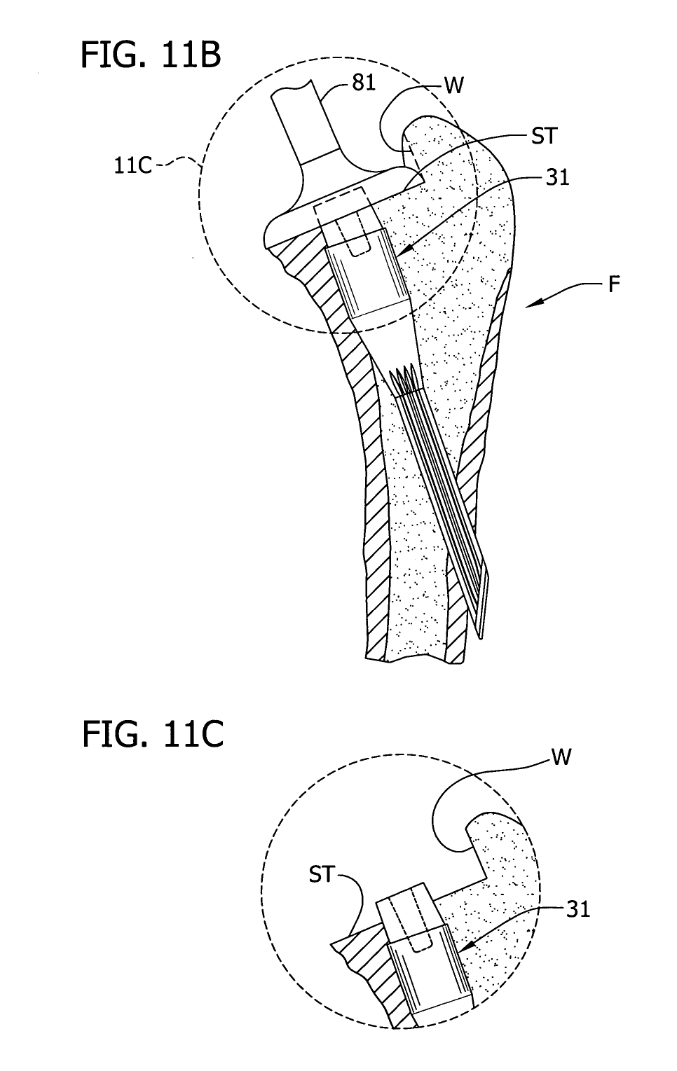

The lip 41 extends from the lateral edge 36 of the collar 25, and may have a suitable radius at its base, such as between about 0.5 and 1.5 mm. The lip 41 is sized and shaped for an interference fit with a wall W (see FIG. 11C) of the seat ST. The lip 41 extends from the upper (proximal) part of the lateral edge 36 to decrease the risk of bone fracture, e.g., fracture of a corner of the femoral seat upon prosthesis impaction. The lip 41 is also beveled to further reduce the risk of fracture. It is contemplated to include multiple lips on the lateral edge 36, and the lips may be of any suitable shape designed to engage the seat ST in the femur F.

As shown in FIGS. 2-5A, three bone graft slots 45 (broadly, openings or pockets) extend inward from the lateral edge 36 for receiving bone graft material (not shown). Upon implantation, the graft material encourages femoral bone (e.g. femoral neck bone bordering the seat) to grow into the collar 25. Thereafter, the grown-in bone functions as an additional interlock between the collar 25 and the femur F to inhibit distraction. Other openings may be used instead of or in combination with the slot including, for example, the vertical grooves shown in the '915 patent. Also, the lateral edge 36 includes three vertical splines 47 that extend outward from between the slots 45 for engaging the wall W of the femoral seat ST. It is also contemplated that the lateral edge 36 of the collar 25 may have more or fewer splines 47 than illustrated herein.

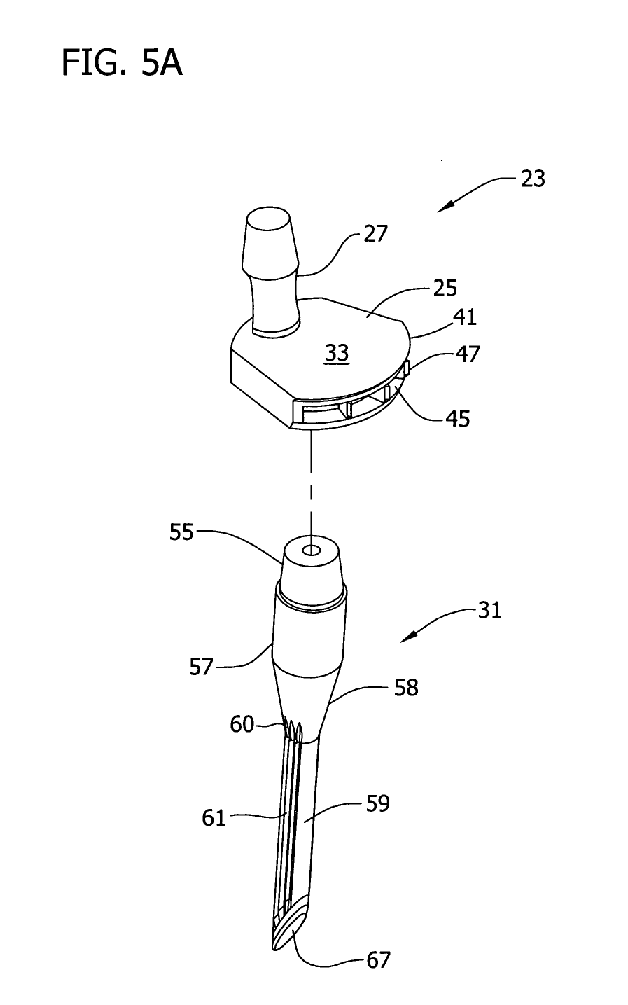

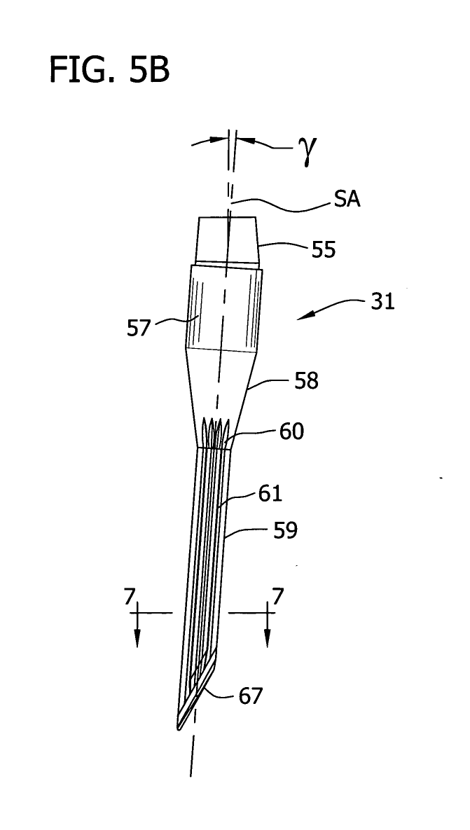

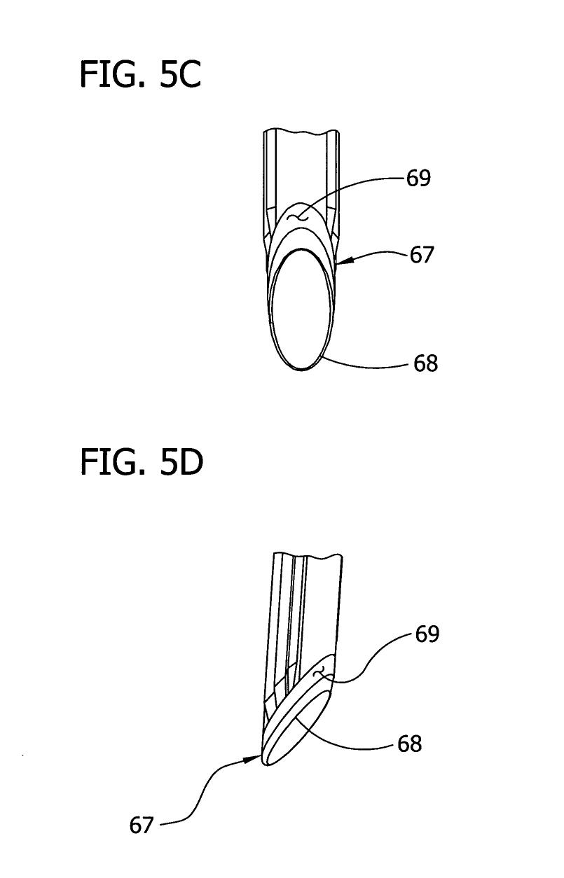

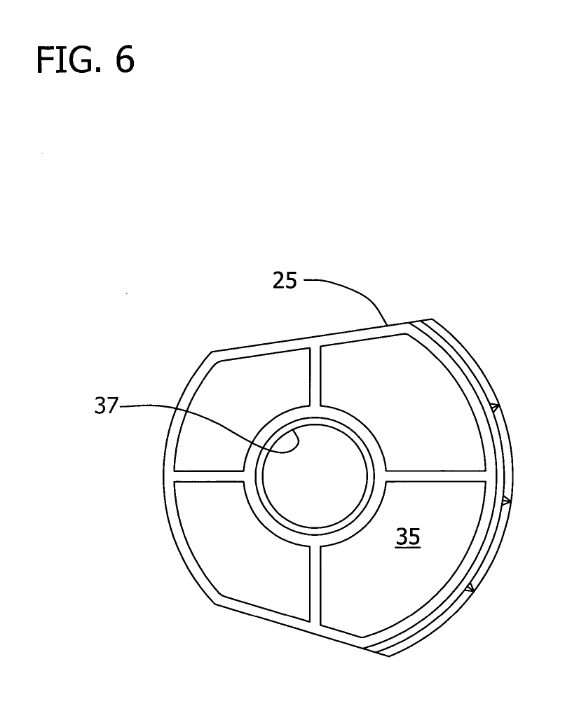

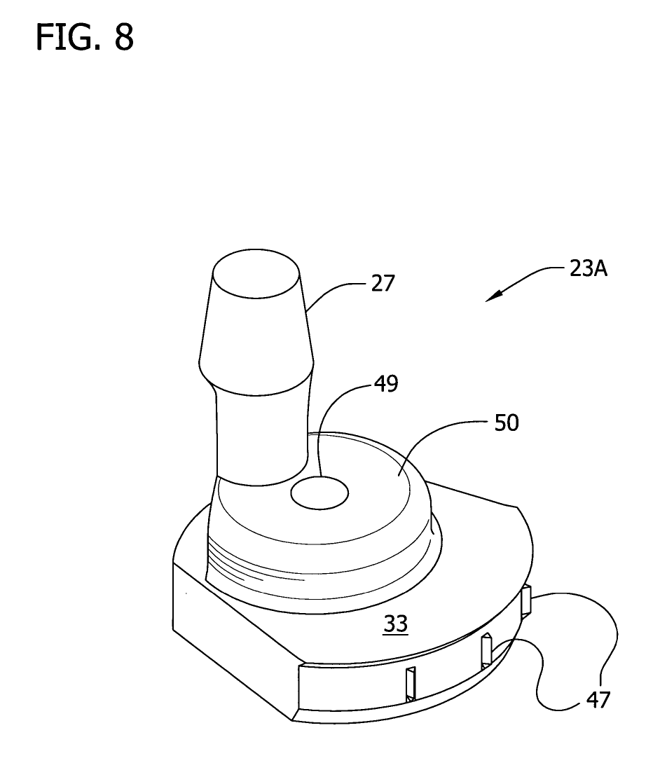

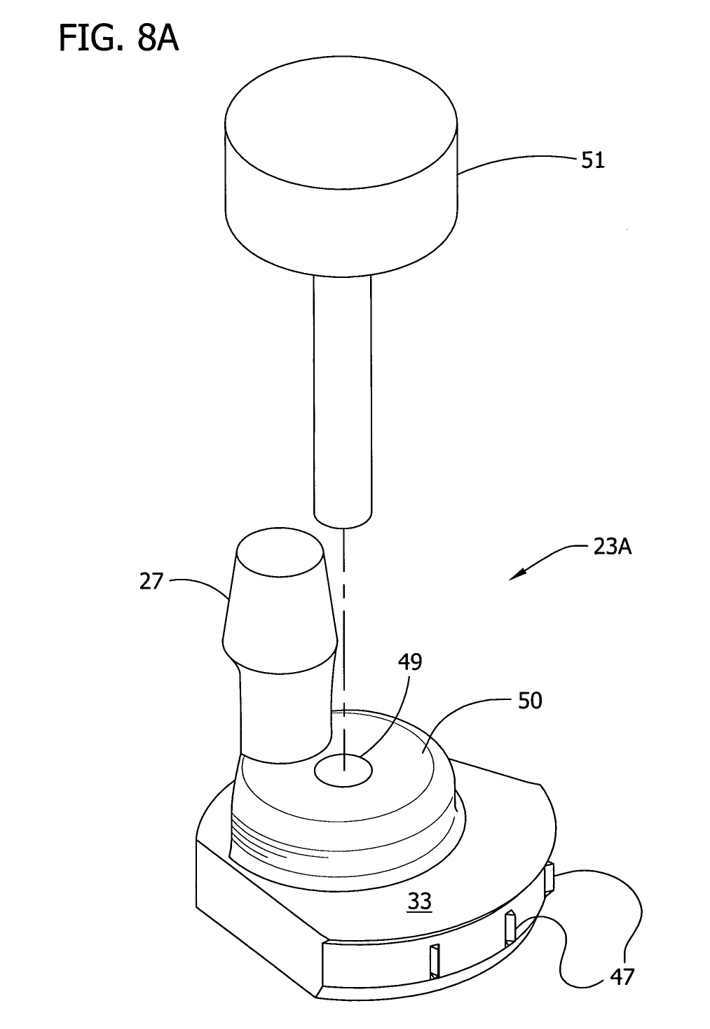

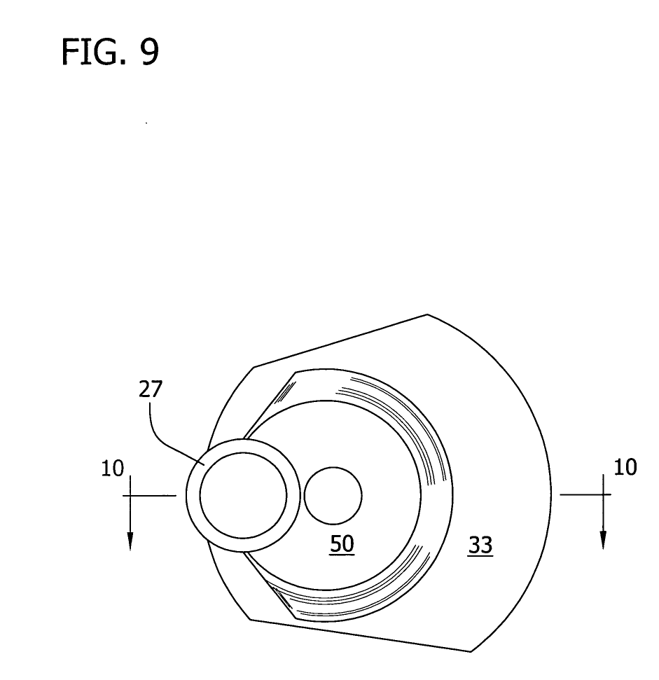

In an alternative embodiment 23A shown in FIGS. 8-10A, the upper surface 33 of the collar 25 includes a cylindric extension 50 so that opening 37 is deeper and may receive more of a mating portion 55 of the stem (FIG. 5B). Thus, the collar 25 is more rigidly fixed to the stem 31 after implantation. Unless otherwise specified, the various embodiments of the prosthesis disclosed herein may be formed with the same elements, subelements, and/or variations as another embodiment described, illustrated, and/or incorporated herein. For example in this embodiment, there are no bone graft receiving openings or structures, though such may be added.

A hole 49 in the extension 50 is sized and shaped to accept an impactor 51 to assemble the modular components (e.g., the stem and the collar) of the prosthesis (FIG. 8A). The hole 49 narrows to allow the impactor to be used to disassemble the stem from the collar, i.e., for test parts that may be used in multiple femurs. The hole 49 may also be in fluid transmission with a cannulated stem, such as the stem described in the '120 patent. As described in detail in the '120 patent, the cannulated stem allows pressure relief and percutaneous sampling of joint fluid, among other advantages. Alternatively or in combination with the cannulated stem, a distal tip 67 may include a central cannula for use in the method described below.

Referring to FIGS. 5A-5D and 6, the stem 31 includes the mating portion 55 for reception in the collar 25 (or the collar of other embodiments), a proximal portion 57, a central portion 58 and a distal portion 59. In this embodiment, the mating portion 55 has a tapered, conical shape and is sized for reception in the opening 37 of the collar 25. The mating portion 55 is angled relative to the stem axis SA at an angle γ. In this embodiment the angle is 4°, but the angle may range from about 1 to about 10°. The angling of the mating portion causes the stem 31 to extend from the collar 25 at an angle β(FIG. 3, β=90°−γ).

The proximal portion 57 of the stem 31 extends generally from the lower surface 35 of the collar 25 as installed. The proximal portion 57 is smooth, not splined, and most of the central portion 58 is likewise smooth, though it suitably includes grooves 60 adjacent its lower end as shown. The proximal portion 57 is cylindrical and the central portion 58 is conical or tapered as shown, though other shapes are contemplated within the scope of the invention. The stem 31 is suitably formed as a one-piece integral assembly, though it may be formed as separate pieces.

The distal portion 59 has splines 61 that can penetrate the femur F around the bore B through the posterolateral femoral cortex to ease insertion of the prosthesis 21 and to inhibit fracture of the femur. The splines 61 have an interference fit with the bore B of the femur F to thereby hold the prosthesis 21 securely against rotational movement about the stem axis SA after implantation, and encourage bone growth around the splines. However, although the splines 61 resist axial displacement of the prosthesis 21 relative to the femur F, the splines do not rigidly fix the prosthesis against axial micromotion.

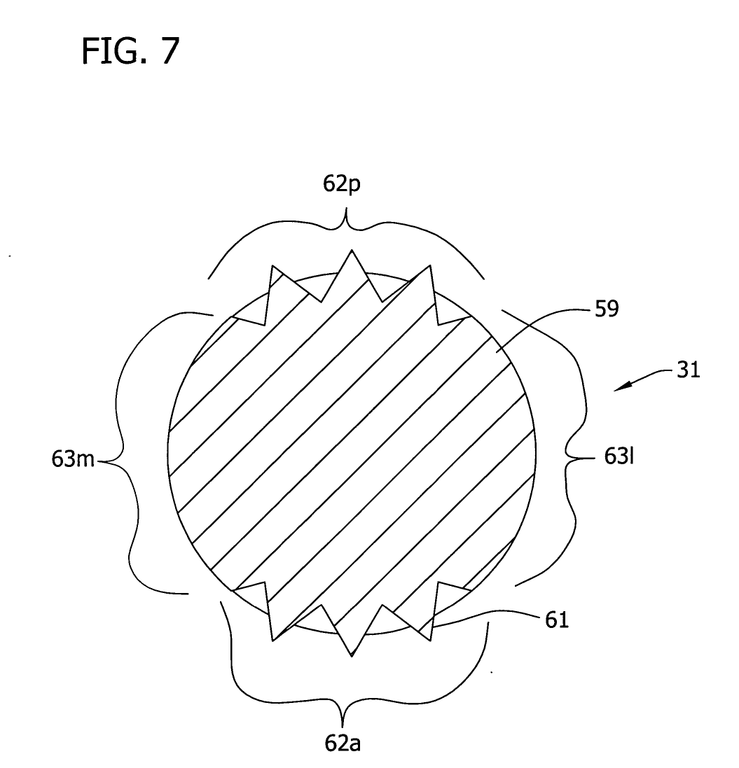

As best shown in FIG. 7, a circumference (broadly, outer periphery) of the distal portion 59 has splined sections 62a, 62P on its anterior and posterior sides, respectively and separated by non-splined sections 63m, 631 on its medial and lateral sides. In this embodiment, each splined section includes three splines having an included angle of 37.5°, a major diameter of 9.00 mm (the outward tip of the splines) and a root diameter of 5.79 mm. Note the diameter of the non-splined sections is about 8.00 mm. The splined sections extend along the distal portion 59 for a distance between about 30 mm and about 60 mm, but the distal tip 67 is not splined as described below. It is understood that the dimensions provided herein are exemplary only and that the stem 31 may have different sized and shaped splines 61. It is also understood that the stem 31 may have more or fewer splines 61.

As described above, the splines along the lateral side or medial side may interfere with proper alignment. In this embodiment, there are no splines along the lateral or medial sides. For example, at least about one-third of the outer periphery of the distal portion is non-splined and generally smooth. The splined and non-splined configuration of the distal portion 59 of the stem 31 inhibits deviation, improves alignment and thereby reduces the risk of assembly or impaction fracture. The configuration also increases rotational stability of the stem 31.

Referring to FIGS. 5B and 5D, the distal portion 59 includes the distal tip 67, which is at an angle to the longitudinal stem axis SA. The angle is selected so that the distal tip 67 is generally aligned (i.e., coplanar) with or parallel to the outer surface of the femur F on the posterolateral side. The tip 67 has a smooth (non-splined), curved (radiused or frustoconical) leading edge 68 and a smooth, cylindrical section 69 adjacent the leading edge. The tip 67 has an acerate (needle-like) shape so that the tip appears pointed as shown in elevation in FIGS. 2-3 and 5B due to its angle relative to the stem axis SA. The tip is suitably angled at between about 15° and about 45°, and in one embodiment is about 25° for an angle of implantation of about 1550. The tip 67 is thus sized and shaped for facilitating insertion of the tip through the entrance and through the exit of the bore. The tip 67 centers the stem in the bore as the prosthesis is impacted or driven into the bore. As implanted (FIG. 2), the distal tip 67 and the distal ends of the splines 61 extend outwardly from the posterolateral side of the femur F to inhibit bone growth over the tip 67 and spline ends. Such bone growth would undesirably fix the prosthesis in an axial direction and prevent the natural loading at the upper end of the femur by the collar.

In this embodiment, the distal tip 67 is formed integrally with the stem 31 so that no lateral incision is necessary to remove the tip. In other words, only one incision is necessary to implant the prosthesis 21.

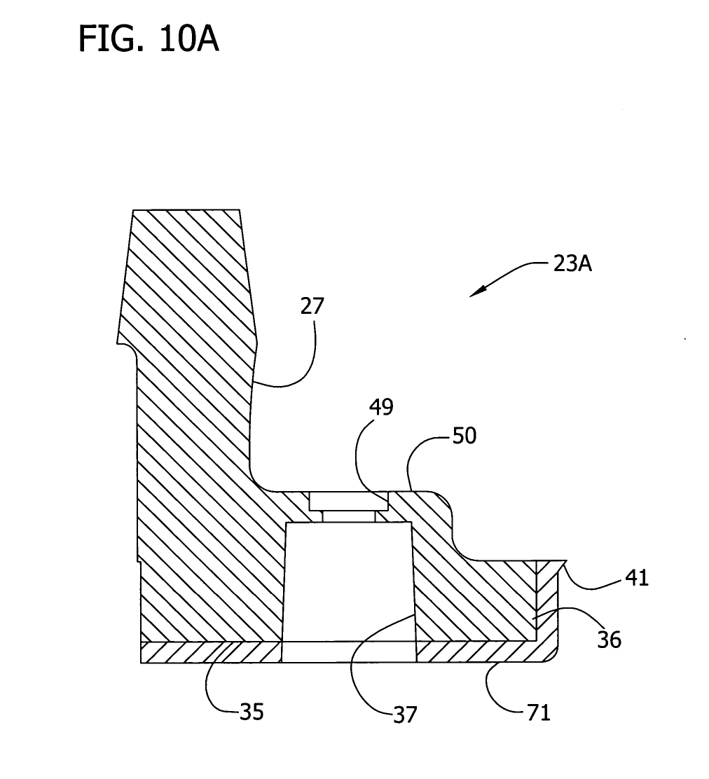

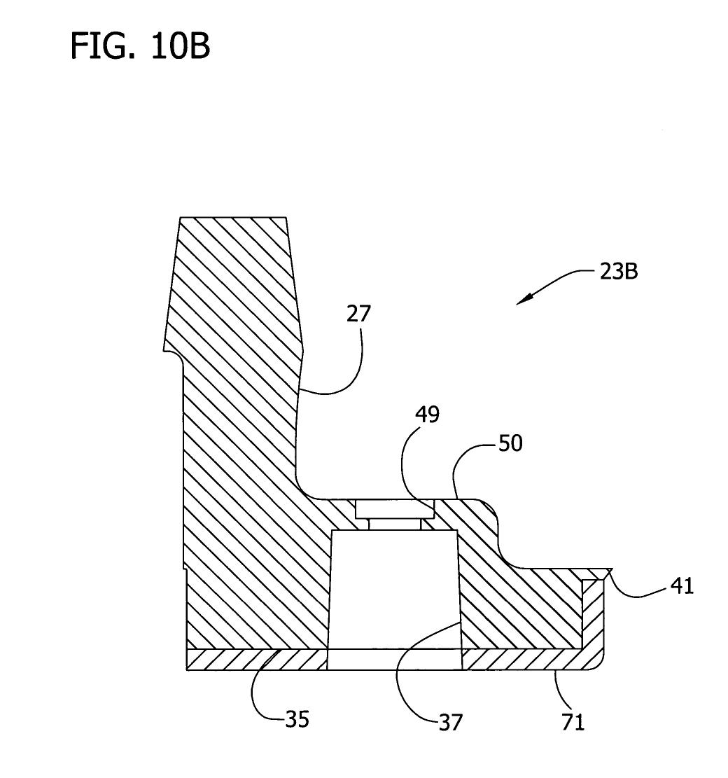

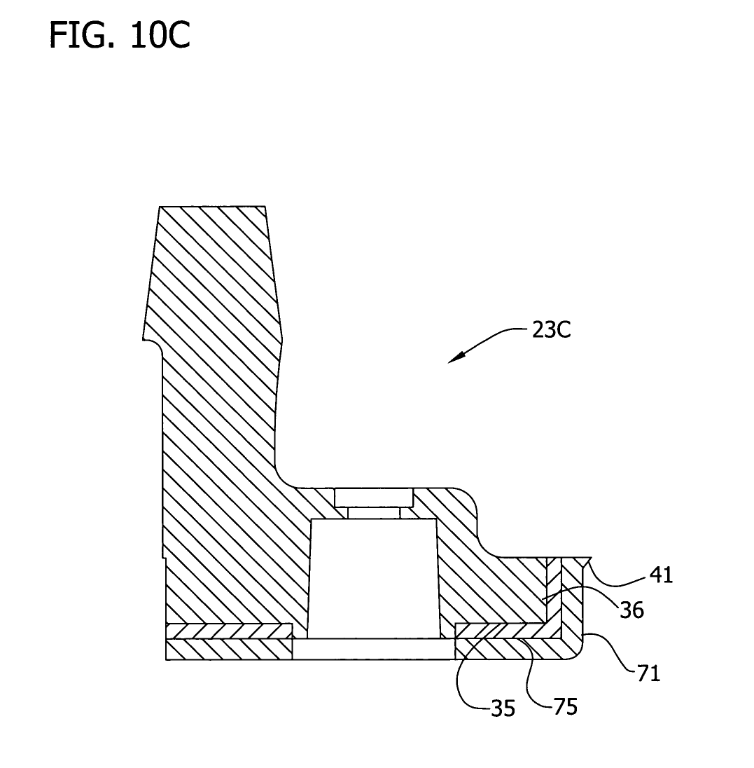

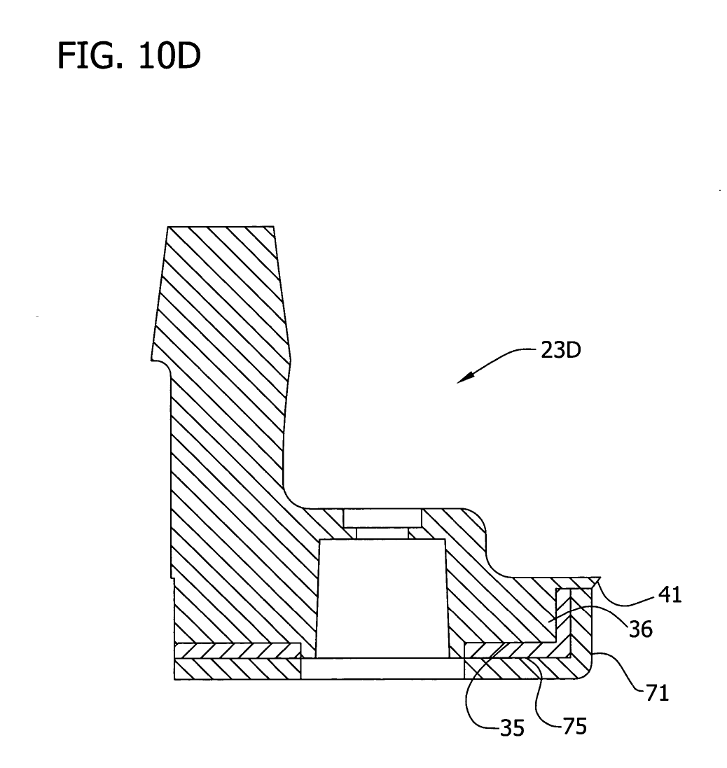

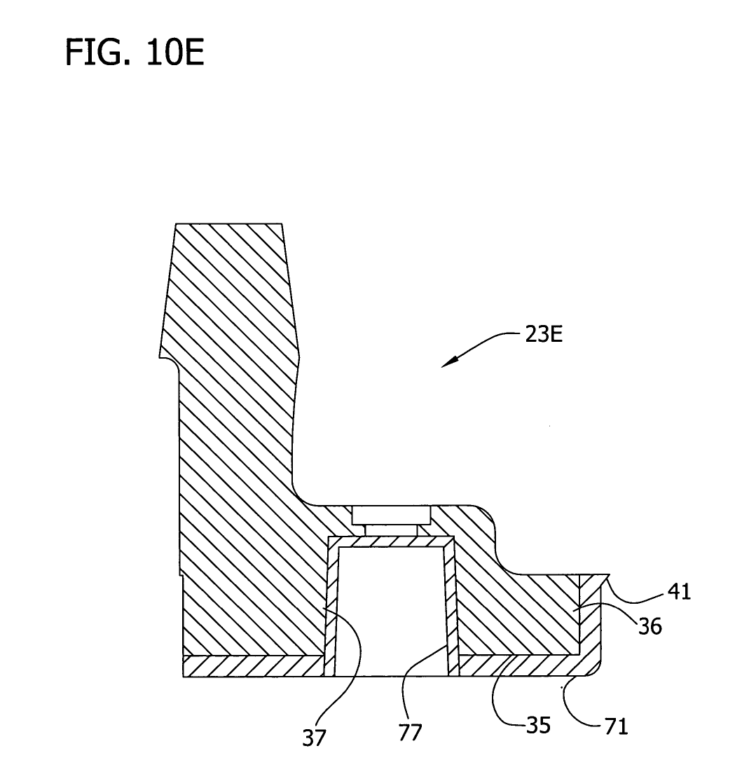

Generally, the prostheses of the invention are made of cobalt-chrome, titanium or other suitable material. Referring to FIG. 10A and collar-neck assembly 23A, the lower surface 35 of the collar 25 may optionally include a “bioactive surface” or porous coating 71, such as porous titanium, porous biomaterial, or sintered cobalt-chrome beads, which promotes bone growth into the coating after implantation, as described in more detail below. The bone growth into the coating inhibits motion of the collar relative to the bone. Note the coating may be heat treated.

Such porous coating 71 may be used instead of (or in addition to) the bone graft slots 45 on the lateral edge 36 of the collar 25. The porous coating 71 may increase friction against cancellous bone and increase initial implant stability.

The porous coating 71 may be applied to the lower surface 35 of the collar 25 in a variety of ways. In assemblies 23A, 23B, the collar is constructed of cobalt-chrome (Co—Cr) and porous coating 71 is applied to the lower surface and the lateral edge (FIGS. 10A, B). The lip 41 on the lateral edge 36 of the collar 25 may be constructed of the porous coating 71 (FIG. 10A) or of the substrate material (FIG. 10B). Advantageously, the stem 31 is made of the same Co—Cr material so the mating parts are constructed of the same metal. The superior strength of Co—Cr also permits a smaller neck 27 cross-sectional area. Note that the stem 31 is suitably constructed from cobalt-chrome alloy to achieve satisfactory distal spline sharpness.

Alternatively, and as shown for assemblies 23C, 23D in FIGS. 10C and 10D, a subplate 75 made of, e.g., titanium alloy (shown in FIGS. 10C, D) is fit over the lower surface 35 and lateral edge 36 of the collar 25. The subplate is suitably press-fit on the Co—Cr collar and porous coating 71 is applied to the subplate 75. In FIG. 10C, the porous coating is used to form the lateral edge lip 41, but in FIG. 10D, the lip is formed in the Co—Cr substrate. The lip 41 may also be formed in the subplate 75. It is contemplated that the subplate 75 may be made from other suitable materials besides titanium alloy.

In assembly 23E shown in FIG. 10E, a sleeve 77 is included in the opening 37 in the lower surface 35. For example, the sleeve 77 is made of Co—Cr and the substrate is a titanium alloy. The sleeve 77 is advantageously made of Co—Cr where the stem 31 is also made of Co—Cr so that the junction is formed in similar metals. The sleeve 77 is tapered, e.g., frustoconical in shape and similar to a conventional unipolar adapter for an endoprosthesis. The sleeve 77 is suitably pressured into the opening in the lower surface of the titanium collar module under high pressure (e.g., five tons). A porous coating is then applied to the titanium substrate. In the embodiment of FIG. 10E, the lip 41 is formed in the porous coating. The lip 41 could also be formed in the titanium alloy substrate. Note that the lip 41 need not be as sharp as the stem splines 61, thereby permitting the use of titanium alloy. As will be understood, a sleeve could be used in any of the embodiments herein.

Note that the coating is typically applied under heat or a combination of heat and pressure. In contrast, the stem 31 of this embodiment is not heat treated to inhibit warpage. The machining of the splines 61 causes residual stress, which may result in warpage of the stem 31 under heat treatment.

Due to the magnitude of forces transmitted through a relatively small area, it may be desirable to increase the strength of the coating, which can be achieved, for example, by increasing the size of the cobalt-chrome beads and/or increasing the number and pattern of reinforcing ribs on the lower surface 35 of the collar.

A method of an embodiment for implanting the prosthesis assures close replication of normal loading of the femur F (i.e., loading prior to implantation of the prosthesis). A femoral head-neck prosthesis that fails to replicate normal loading conditions will change the stress distribution through the femur F. As mentioned in the '937 patent, according to Wolff's law these changes in stress distribution eventually cause alterations in the internal structure of the bone. Those portions subject to a lesser stress than before are likely to deteriorate and those subject to greater stress than before are likely to thicken. But if the stress is too great and applied over an extended period, bone cells may be killed. To replicate normal loading, the method of the present invention aligns the stem of the prosthesis with the average compression loading vector for the particular femur, which vector is variable from person to person. The prosthesis may be suitably implanted in a manner similar to one of the implantation methods shown and described in the '120, '915 and '937 patents.

One method of implantation is a single incision anterior approach, a form of minimally invasive surgery (MIS). This approach has many advantages. The distal tip 67 of the stem 31 described above, in conjunction with the anterior approach, eliminates the need for an additional incision adjacent the bore exit through the lateral femoral cortex. Moreover, the placement of the anterior incision, combined with externally rotating and extending the femur F, conveniently tends to direct the bore B and the stem 31 toward the posterolateral femoral cortex. The approach allows for excellent acetabulum/femur visibility. It is an internervous approach and does not require cutting, splitting or dividing muscles, which can result in irreparable harm to the muscles. The approach promotes normal hip mechanics, immediate hip stability and reduced dislocation risk. Accordingly, it eliminates the need for postoperative immobilization and restrictions on hip motion. There is less tissue trauma, less blood loss, less postoperative pain, and pain medication. In general, the approach enables a faster recovery and fewer restrictions on postoperative activities.

When using the single incision anterior approach, an incision of approximately 7 cm to 10 cm in length (depending on the size and anatomy of the patient) is made between adjacent the anterior superior iliac spine and a point anterior to the tip of the greater trochanter with the patient in a supine position. The interval between the tensor facia lata muscle and the sartorius muscle is developed (i.e., made deeper and/or wider) and the anterior hip capsule is identified and incised. Retractors are then placed around the femoral neck. Next anatomic landmarks, such as locations on the pelvis and femur, are registered and stored in the memory of a computer positioning system. A hip skid is placed around the femoral head and the hip is dislocated by traction and external rotation. Additional anatomic landmarks, for example, on the femoral head and neck are registered to help localize the anatomic center of the femoral head. The MTS axis, which was radiographically determined before surgery, is displayed along with the femoral neck resection plane on a monitor of the computer.

A saw guide used for resecting the femoral head and a portion of the neck is positioned with computer assistance to a proper orientation with respect to the MTS. Once the saw guide has been properly placed, a saw is used to remove the femoral head and a portion of the neck. With computer assistance, the acetabular component is installed.

Next, the lower extremity of the patient is dropped toward the floor and externally rotated such that the foot is pointing outward. Retractors are placed around the proximal femur to elevate it toward the incision. In some instances, capsular bands need to be released to mobilize the proximal femur.

With the femur in an extended and externally rotated position, the position of the incision and muscle exposure is approximately aligned with the desired axis for creating the bore. The femoral neck is reamed with computer guidance keeping the reamer appropriately oriented with respect to the MTS. A guide is pressed into the reamed femoral neck and a guide sleeve is passed through the guide. A guide pin (not shown, see '915 and '120 patents) attached to a power drill is passed through the guide sleeve at the desired angled in relation to the MTS, which is determined using computer guidance. Using the drill, the pin is passed through the posterolateral femoral cortex. The pin alignment is reconfirmed in regard to degrees of anteversion and the angle between the pin and the lateral femoral cortex. If the pin is not properly aligned, the pin can be repositioned using the steps provided below. If the pin is properly aligned, the sleeve and guide are removed. The guide is placed over a cortical drill-reamer, and the reamer is passed over the guide pin. Using the power drill, the reamer is used to form the bore.

The proximal femoral seat is planed to the desired depth using computer assistance, trial implants are inserted, and the hip reduced (i.e., the ball is placed into the acetabular component). Computer assistance is again used to check restoration of leg length and femoral offset (i.e., horizontal distance between center of hip rotation and the femoral shaft).

Suitable computer systems include Surgical Navigational Technology, including Mini-Incision Hip Navigation, available from Medtronic of Minneapolis, Minn., VectorVision® Exactrac available from Brainlab AG of Munich, Germany, and Stryker® Navigation System available from Stryker Corporation of Kalamazoo, Mich. Suitable systems can use image-based or position-based tracking.

The method uniquely combines bone preserving technology (the transosseous prosthesis) with minimally invasive approaches and with computer assisted surgery. Bone preservation results in better outcomes and reduces the likelihood of implant failure. It will be understood that other approaches might be used, such as any standard hip replacement surgical approach, and including any minimally invasive approach.

As discussed above in the Background section, in most cases the stem cannot be aligned exactly with the MTS axis for anatomical reasons, e.g., because the axis extends through the medial neck cortex above the lesser trochanter. Previous transosseous implants, such as the implant shown in the '120 patent, were installed in “offset” alignment so that the stem was parallel to the MTS axis, e.g., offset from the axis so that the bore did not extend through the neck cortex. A disadvantage of “offset” alignment is that it creates a bending moment on the stem that may excessively strain parts of the femur F, e.g., the femoral neck N.

In one embodiment of this invention, the stem 31 is implanted on an “intercept” alignment shown in FIGS. 2 and 11A-B. The bore B through the femur F is more vertical than the MTS axis (i.e., it forms an acute angle therewith) so that the proximal portion 57 of the stem 31 is offset from the MTS axis but the distal portion 59 exits the bore at a point on the MTS axis, or so that the stem axis SA is within about 10 mm of such point. The bore B must be formed so that its exit through the lateral femoral cortex is disposed at the point on the MTS axis. The bore may suitably be formed for intercept alignment using the computer positioning system to precisely position the guide pin in three-dimensional space so that the bore extends at the proper angle relative to the MTS axis. It is important in intercept alignment to ensure that the bore B is properly aligned since even a few degrees of variance makes a significant difference in the loading or strain placed on the bone. The use of the computer system is advantageous compared to other methods. However, it is contemplated to use another system, such as those disclosed in the '120 and '915 patents.

The “intercept” alignment decreases the distance between the MTS axis and the stem 31 and thereby decreases the bending moment on the stem. Moreover, intercept alignment causes the bore B through the femur F to be longer than an offset alignment bore so that the stem contacts more femoral bone, especially the lateral cortex. The increased contact area between femur F and stem 31 should increase the torsional stability of the prosthesis.

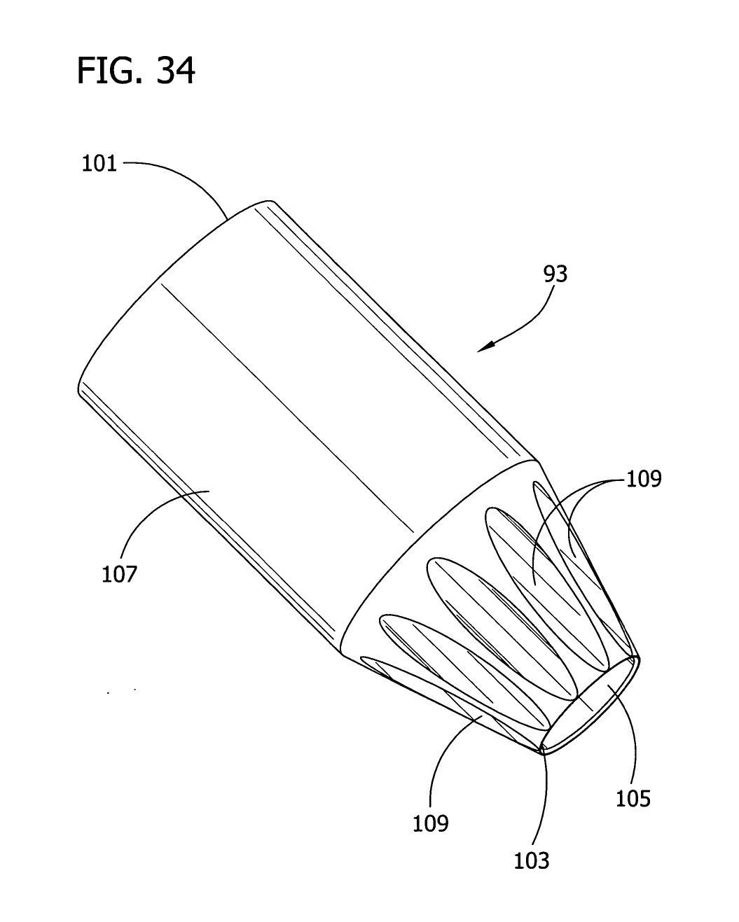

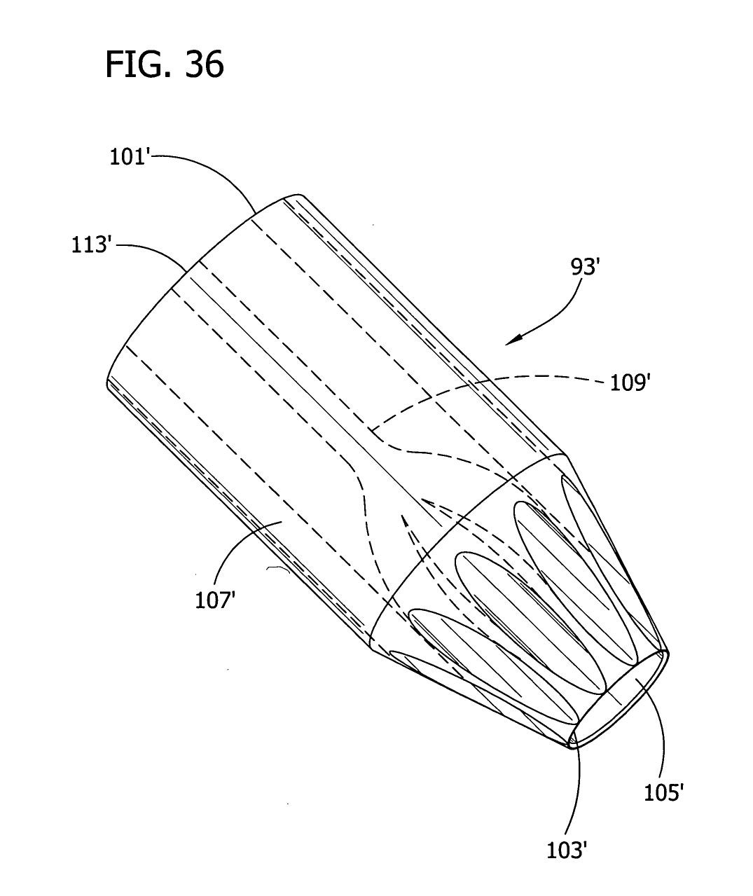

Due to the increased length of the bore B and the density of the surrounding cortical bone C, there is an increased risk of thermal necrosis of the femur F during reaming of the bore. Accordingly, a cooling system may be used during reaming. For example, a fenestrated guide, indicated at 93 in FIGS. 34-36, has fluid passages and may be used around the reamer shaft 95 having a reamer 97 mounted thereon for directing cooling fluid 99 (e.g. saline) to the femur F during reaming. The guide 93 is used to align the shaft 93 extending from a drill (not shown) having the reamer 97 thereon during the implantation of the prosthesis 21. The guide 93 comprises a cylindrical body having a top 101, a frustoconical shaped bottom 103, an inner wall 105, and an outer wall 107. The inner wall 105 defines a cylindrical opening for allowing the shaft 95 to slideably pass therethrough. The guide 93 is generally sized and shaped for insertion into the upper portion of the bore through medullary canal MC that has already been prepared to receive the proximal and central portions 57, 58 of the stem 31. As a result, the guide 93 has a configuration similar to the proximal and central portions 57, 58 of the stem 31, which includes the cylindrical body and a generally frustoconical shaped bottom 103.

Ten passages 109 extend between the inner and outer walls 105, 107 from the top 101 to the bottom 103 of the body for allowing fluid 99 to pass through the guide 93 for direct fluid contact with the reamer 97 and the cortical bone C being cut by the reamer to thereby cool the reamer and bone. In the illustrated embodiment, the passages 109 include a plurality of fluid inlet passages for allowing fluid 99 to pass from the top 101 of the body to the bottom 103 of the body, and a plurality of outlet passages for allowing fluid to pass from the bottom of the body to the top of the body. It is understood that any of the passages can be used as either an inlet passage or an outlet passage.

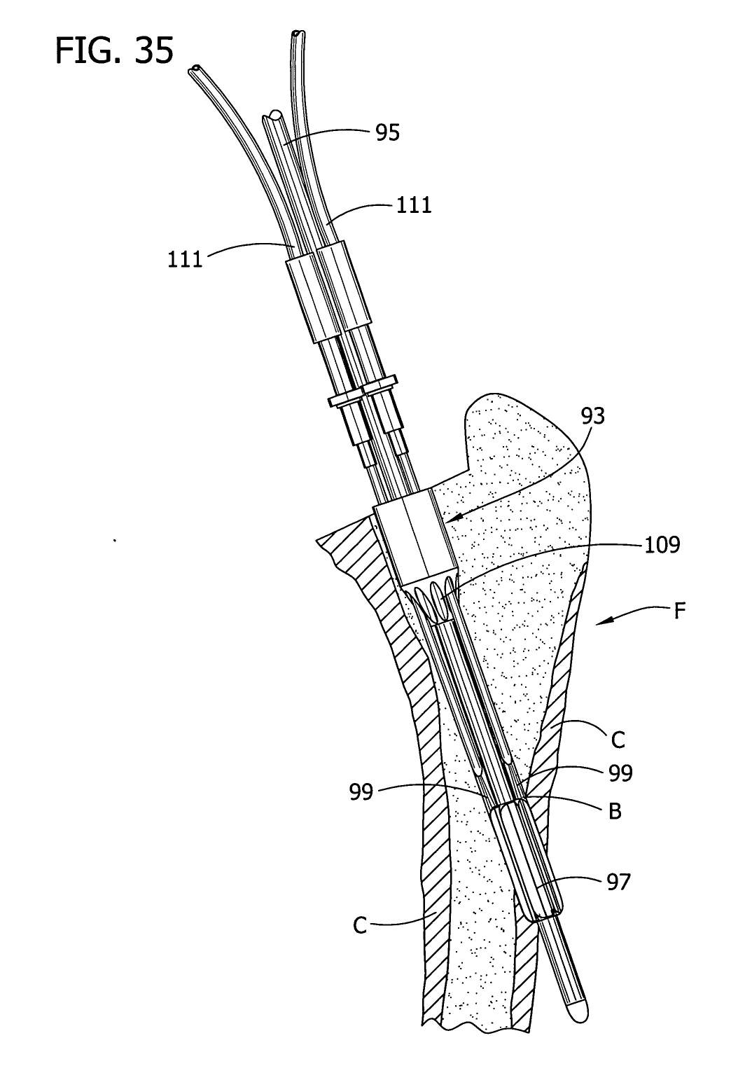

With reference to FIG. 35, catheters 111 are inserted through the inlet ports. One end of the catheters are connected to a pump (not shown) for pumping cooling fluid 99 through the catheters and out the opposite end of the catheter, which is located in the bore B in close proximity to the reamer 97. The outlet passages allow gas and/or fluid to be exhausted from within the bore B to thereby prevent pressure from building up within the bore when the cooling fluid 99 in being pumped into it. It is also understood that the guide may have more or fewer passages than the illustrated embodiment.

In another embodiment (FIG. 34A), the guide 93′ has a single channel 113′, which is located on the top 101′ of the guide, fluidly connected to the inlet passages 109′. The single channel 113′ allows a single catheter (not shown) to be used for supplying cooling fluid to a plurality of passages 109′.

After reaming, the stem 31 is driven into the bore so that the splined sections 62a, 62p on the anterior and posterior sides of the stem engage the bone on the anterior and posterior sides of the bore B, respectively. The non-splined sections 63m, 631 are disposed inferomedially (broadly medially) and superolaterally (laterally), respectively, and not necessarily in contact with the bone. The splines 61 of the stem 31 bite into the walls of the bore B and the stem protrudes slightly through the oblique exit hole of the bore so that cortical bone C does not later grow over the end of the stem. Growth of bone over the end of the stem 31 would be undesirable since it would impede the ability of the prosthesis 21 to transmit loads from the hip to the upper femur.

Referring to FIGS. 11A-B, because the stem 31 of this embodiment is angled relative to the collar 25, the seat ST formed in the femoral neck is angled relative to the bore. In this embodiment, the mating portion 55 is angled relative to the stem longitudinal axis SA. In one method of forming the seat, the stem 31 is implanted so that the longitudinal axis of the mating portion 55 is aligned perpendicular with the angle of the seat to be formed (referred to as the anteversion of the femoral neck). A calcar planer 81 having a stud 83 along its central axis is then fitted in the cannula 79 of the mating portion to plane the neck N and thereby form the seat ST. The collar 25 is thereafter impacted onto the mating portion 55 of the stem 31, the angle of the mating portion ensuring that the lower surface 35 of the collar engages the main surface of the seat ST and so that the collar lateral edge 36, including its lip 41, engages the wall W of the seat. As can now be seen, the neck 27 of the prosthesis is adapted for implantation so that the neck axis NA is parallel or co-linear with the medial trabecular stream AX-3. Further, the stem 31 is adapted for implantation so that in its proximal portion 57 the stem axis SA is offset from the medial trabecular stream AX-3 and so that the distal portion 59 intersects the trabecular stream TS. Also, the lip 41 in the collar inhibits “pistoning” of the prosthesis 21 after implantation.

It is contemplated that a temporary axle (not shown) be placed in the cannula of the taper to guide the collar during impaction. Use of such an axle would likely require application of a counterforce to the distal end of the stem 31, and thus necessitate a second incision adjacent the exit of the bore B through the lateral femur. Once the collar 25 is implanted, an appropriately sized ball 39 is locked onto the neck 27. The ball 39 is received in the acetabulum or a prosthetic cup in the acetabulum (not shown).

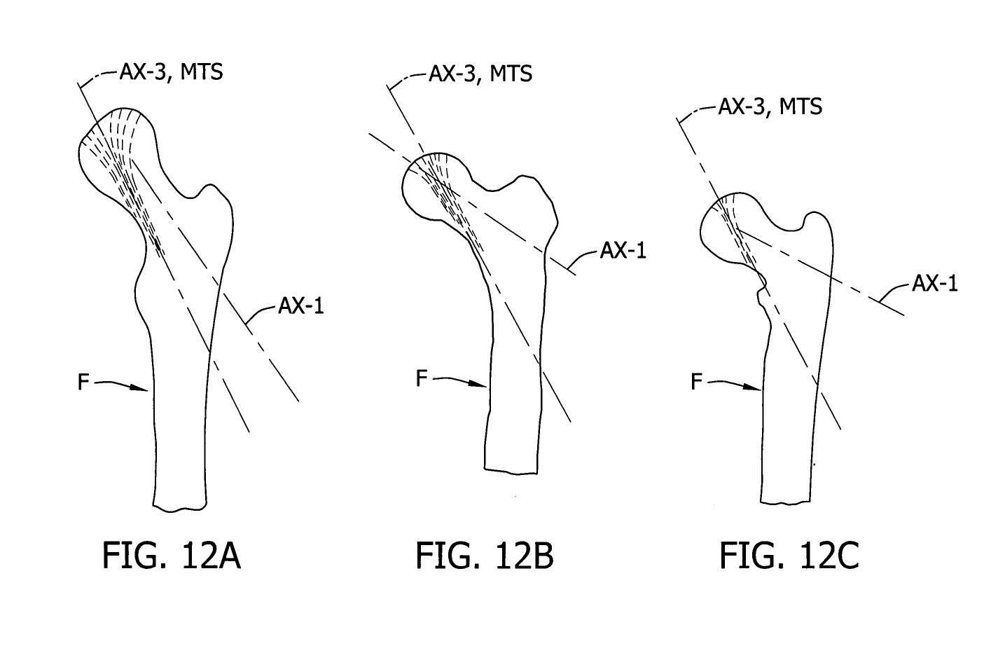

The goal of “axial alignment” is to implant the prosthetic neck axis NA and stem axis SA (or at least the distal stem axis) co-linear with the MTS axis AX-3. Referring to FIGS. 12A-12C, there are different types of femurs F, and it is difficult to implant a prosthetic stem in axial alignment in some types. In FIG. 12A, a valgus femur has a relatively vertical neck axis AX-1 so that the medial trabecular stream AX-3 extends through the marrow (cancellous bone) and the medial femoral neck is relatively thin. An average femur (FIG. 12B) and a varus femur (FIG. 12C) have a more horizontal neck axis AX-1 and more horizontal angle between the femoral shaft and the femoral neck. In average and varus femurs, the medial trabecular stream MTS converges on the medial femoral neck cortex and the bone is thicker and denser.

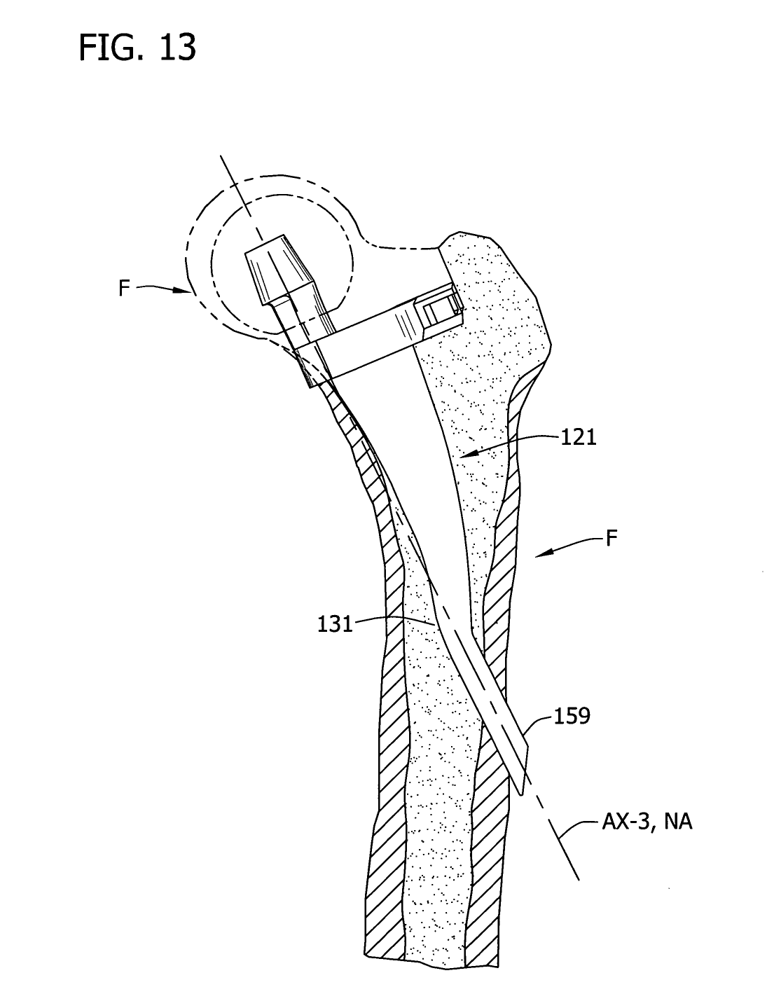

The prostheses and methods of this invention may be modified for axial alignment. For example, a prosthesis 121 shown in FIG. 13 has a “bayonet” or “lazy S” shaped stem 131. The stem curves distally within the medullary canal MC of the femur F to align with the MTS axis. Axial alignment is advantageous in some respects but difficult to achieve due to the need to preserve the medial femoral neck cortex. The shape of the stem 131 offsets the proximal portion of the stem from AX-3 to avoid the cortical bone C and thereby preserve the bone, but the distal portion or portions thereof are co-linear or nearly co-linear with AX-3. This curved stem 131 is most feasible for more valgus femurs such as shown in FIG. 12A. Because the medial trabecular stream AX-3 extends through the medullary canal MC of a valgus femur, and because the medial neck cortex is not as thick in a valgus femur (compared with average or varus femurs), axial alignment is more readily achieved. In other words, the stem 131 is bent around a less extreme ‘corner’ (the medial neck cortex). However, such a stem 131 is typically not practical for average and varus femurs because there is insufficient space medial to lateral within the medullary canal to impact or drive the stem distal portion 159 through the cortical bone C. The bore B for such a prosthesis is suitably reamed from the lateral side of the femur, as described below, which necessitates a second incision. Alternatively, a larger bore could be formed in the femur for receiving a curved stem. Drilling a bore through the medial femoral neck cortex of average and varus femurs would permit axial alignment but would not preserve the medial femoral neck cortex. The stem 131 is one-piece as shown, but may be separable or modular.

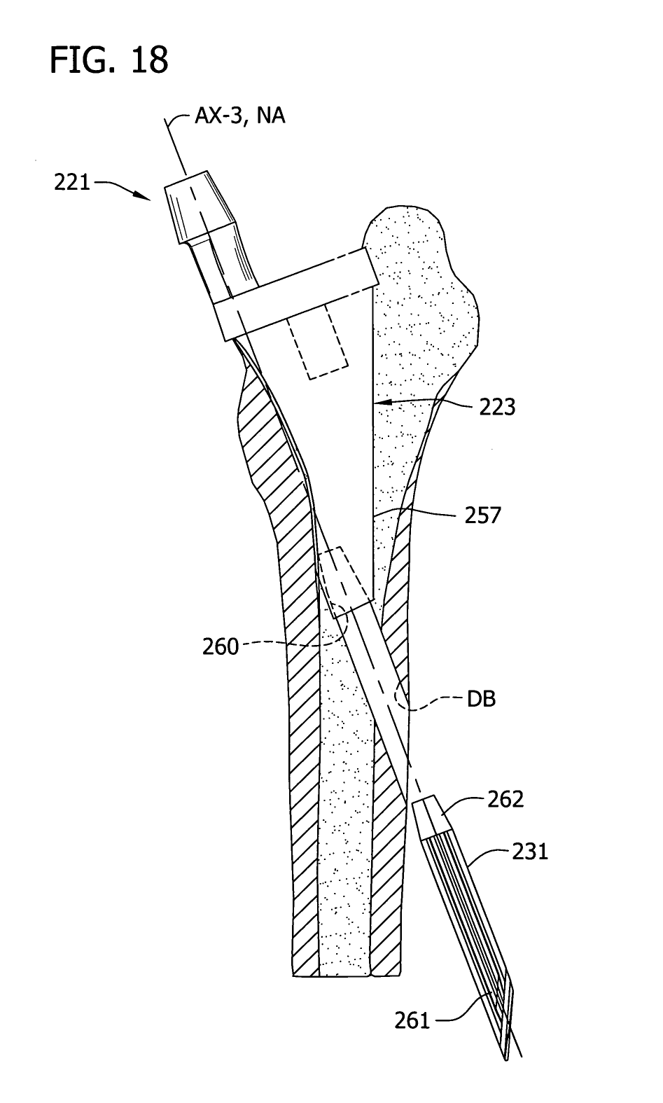

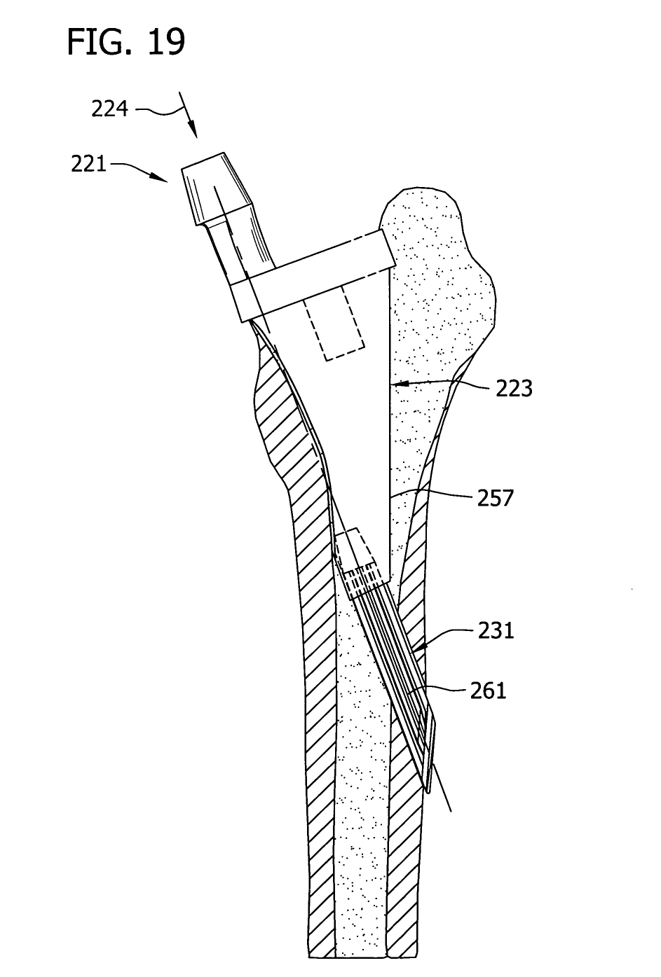

Referring to FIG. 19, a modular retrograde prosthesis 221 comprises a collar-neck-stem assembly 223 that includes the stem proximal portion 257, and separately a distal stem assembly 231. The distal stem assembly includes splines 261 along its central portion 258. A distal end of the stem proximal portion 257 includes a hole 260 (FIGS. 18-19) for receiving a self-locking taper 262 of the distal stem assembly. The collar may be modified to include any of the features described herein.

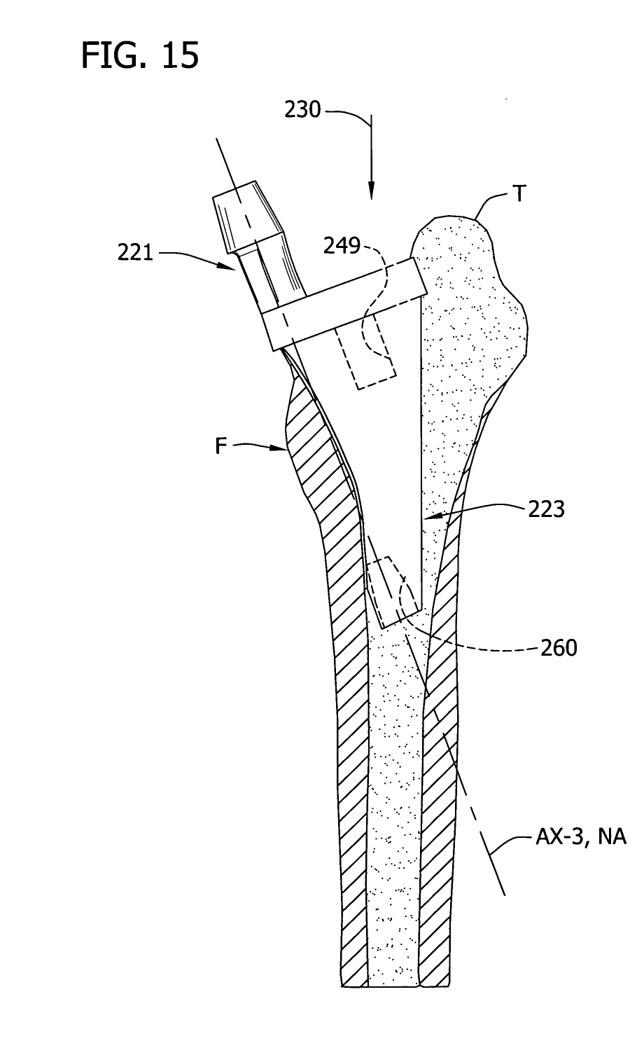

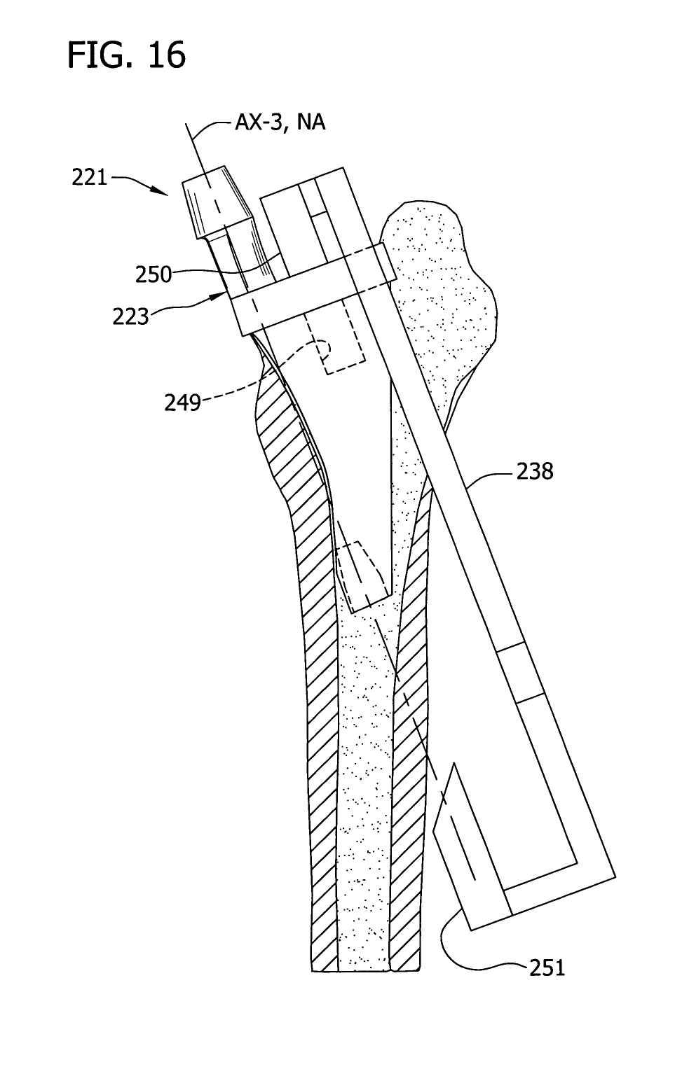

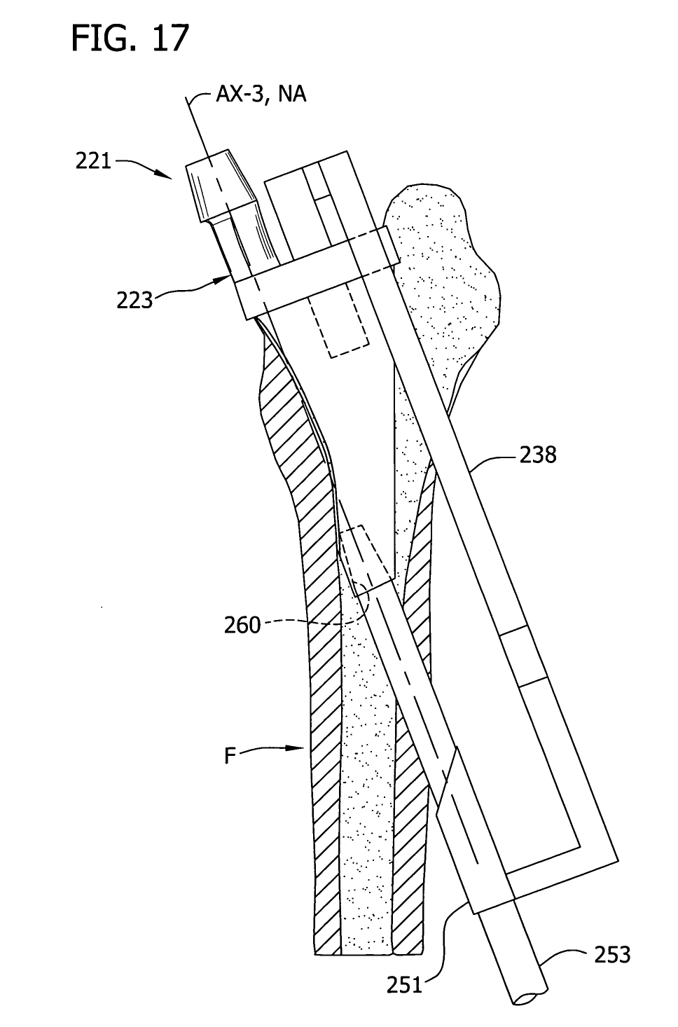

FIGS. 14-19 show a method of installing the prosthesis 221. The femoral neck N is resected and planed to form seat ST shown in FIG. 14. The collar-neck assembly 223 is inserted vertically down the medullary canal MC of the femur F along the direction of the arrow 230 (FIG. 15). An alignment guide 238 includes a stud 250 that is inserted into a socket 249 in the collar-neck assembly (FIG. 16). A drill guide 251 at a distal end of the alignment guide 238 aligns a cortical drill bit 253 with the female self-locking taper 260 at the distal end of the stem proximal portion (FIG. 17). The drill bit forms a distal bore DB (FIG. 18). Note the distal bore DB is drilled retrograde (from distal to proximal) in alignment with the taper 260. The distal stem assembly 231 (FIG. 18) is then impacted or driven through the distal bore DB (FIG. 19) and into the taper 260 of the proximal portion 257 while applying a counter force to the collar-neck assembly 223 along arrow 224.

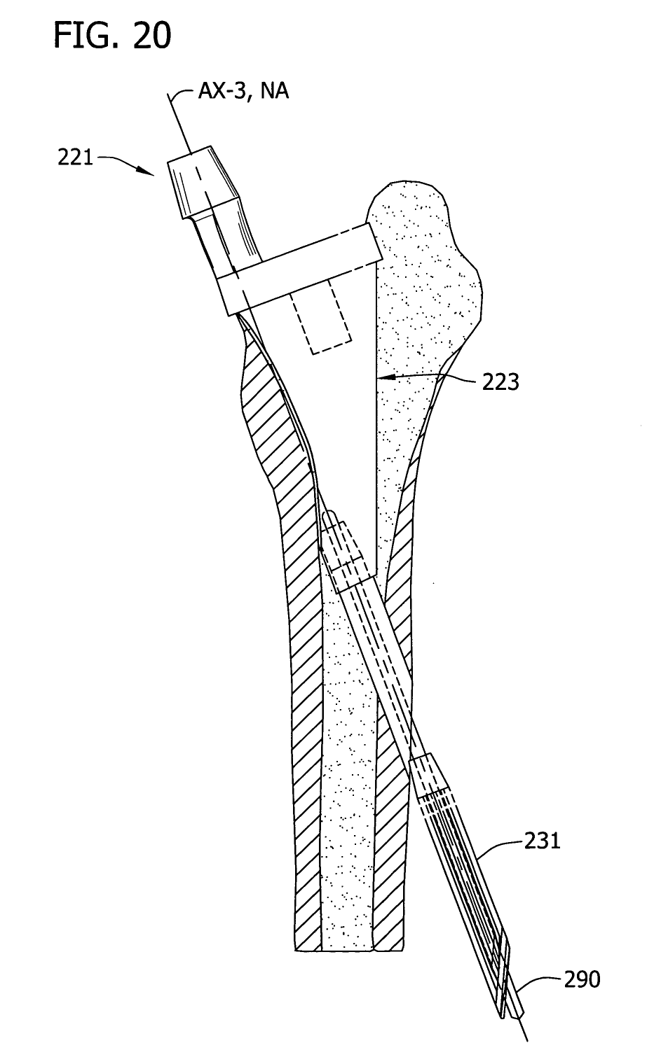

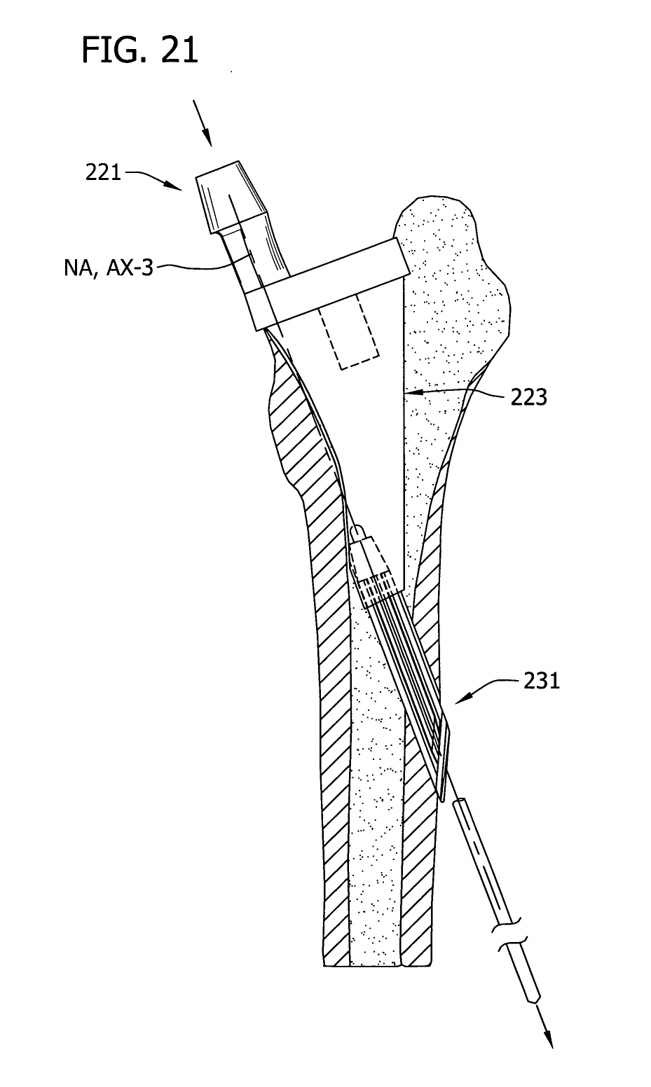

A variation of the method is shown in FIGS. 20-21 and uses a guide pin 290 to align the distal bore DB. The angle of the guide pin 290 may be adjusted using the side-cutting burr and the method described below. The distal stem assembly 231 is cannulated in this variation, and is impacted over the pin into the female taper (FIG. 20). The pin is thereafter removed (FIG. 21).

A retrograde prostheses, such as prosthesis 221, and the associated installation methods achieve the goal of axial alignment of a trajectory-matched, compression-enabling Total Hip Arthroplasty (THA) prostheses for all femurs, regardless of neck angle NA.

Generally, axial alignment has several advantages:
a) The loads experienced by the interface of collar and femoral neck (collar-neck interface) will be predominantly in compression. The goal is to have the average weight-bearing load be perpendicular to the plane of the collar to load the planed femoral neck N in compression. There is a range of loads that a hip experiences, however, the majority of them, and the highest level loads (average of 2.3×body weight during level walking) go through a narrow range of trajectories.
b) Axial alignment will minimize shear forces on the collar-neck interface, which is a corollary of (a). The collar could have a porous coating (as described above) to minimize shear or interface slippage to allow bone and soft tissue to grow into the porous coating to create a stable implant.
c) Axial alignment will optimize translation of the distal portion of the stem through the bore under compression load.
d) Axial alignment will minimize the bending moment on the prosthesis. The lowest bending moment the prosthesis will incur is if the neck axis NA and distal stem axis SA are co-linear. Any bending moment that would occur would then be due to natural variability of loads generated by the hip, rather than an intrinsic bending moment due to prosthesis configuration and alignment. For example, axial alignment would likely minimize the polar moment of inertia exerted on the prosthesis during activities such as stair climbing.
e) Axial alignment places the cortical bore more distally and therefore it extends through thicker and stronger bone. Testing shows such a bore increases the resistance to rotational forces (improved initial stability against torque).

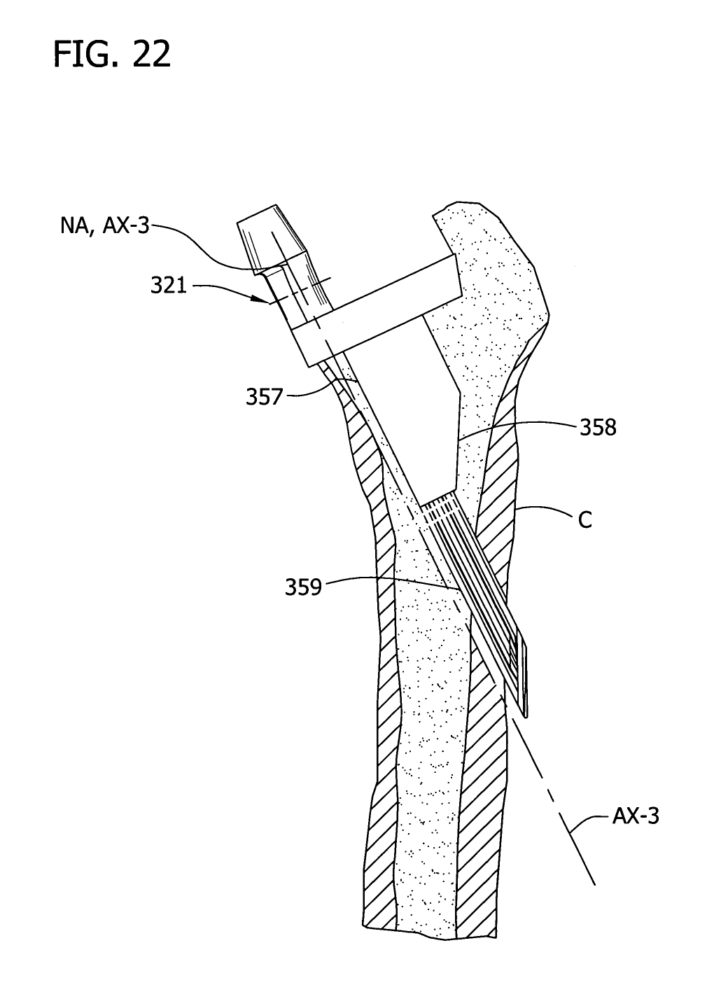

Referring to FIG. 22, another embodiment, prosthesis 321, is implanted parallel to the MTS axis AX-3 but somewhat offset proximally. The stem includes a proximal portion 357 in the shape of a large-diameter cylinder which tapers along its lateral side 358 to a smaller diameter cylinder in the distal portion 359. This stem has less extreme curvature within the medullary canal MC and may be implanted antegrade as in the MIS approach described above. This stem is advantageous in that the distal stem is parallel to the MTS, and is inserted into a bore B that extends through somewhat thicker cortical bone distally. Note the prosthesis 321 may be formed with the same elements, subelements, and/or variations as another embodiment described, illustrated, and/or incorporated herein.

Experiments have demonstrated that horizontal or vertical deviation of the stem axis SA of five degrees or less from the MTS axis AX-3 significantly changed the strain in the proximal femur. A more horizontal alignment increases strain; a more vertical alignment decreases strain. Thus, it is desired to have the horizontal and vertical deviation of the stem axis SA with respect to the MTS axis AX-3 that is less than four degrees, and preferably less than two degrees. Data from laboratory testing conducted on an intact human femur showed that full strain restoration can be closely replicated if the load trajectory was aligned within one degree of the radiographically determined MTS axis. However, when the load trajectory varied from the radiographically determined MTS axis by approximately five degrees, the strain restoration varied significantly. As previously mentioned, too much strain causes bone to thicken, and too little strain causes bone to deteriorate.

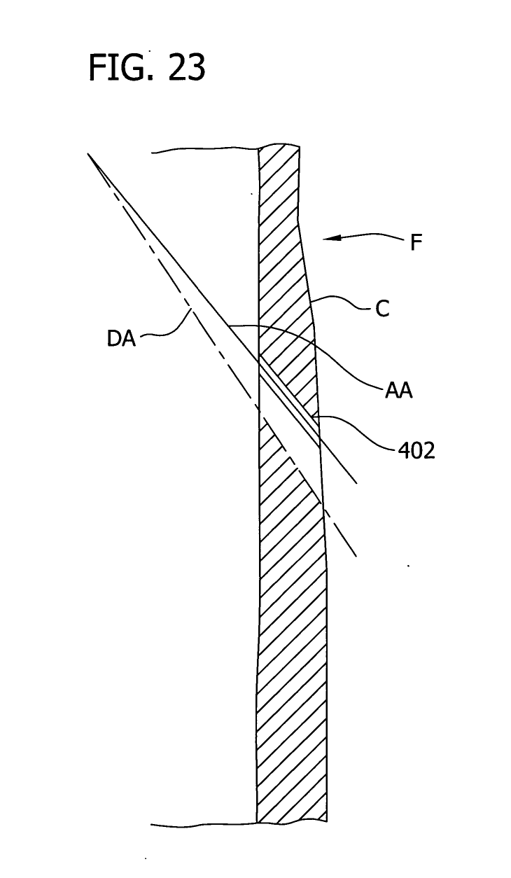

Referring to FIGS. 23-28, instruments and a method of the invention improve the precision with which the bore B is drilled/reamed so as to match the desired angle of implantation. This method applies to among other approaches open surgery (where the lateral femoral shaft is exposed), fluoroscopically guided minimally invasive surgery or computer assisted minimally invasive surgery.

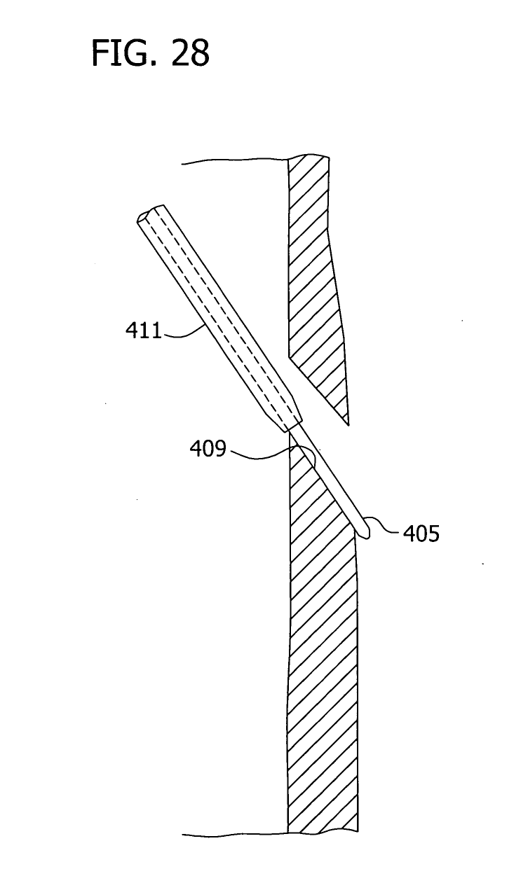

If, after passing a guide pin 405 (FIG. 28) through the guide and lateral femoral cortex, the actual angle is substantially (e.g., >5°) off the desired angle, the guidepin can simply be withdrawn and re-drilled at the desired angle. If the guide pin 405 is close to the desired angle but not optimal (within 4°) it may be difficult to simply re-drill the pin angle. The guide pin tends to drop back into the same hole 402 (FIG. 23), especially due to the acute angle of incidence of the guide pin in relation to the cortex. Accordingly, incremental adjustment is needed.

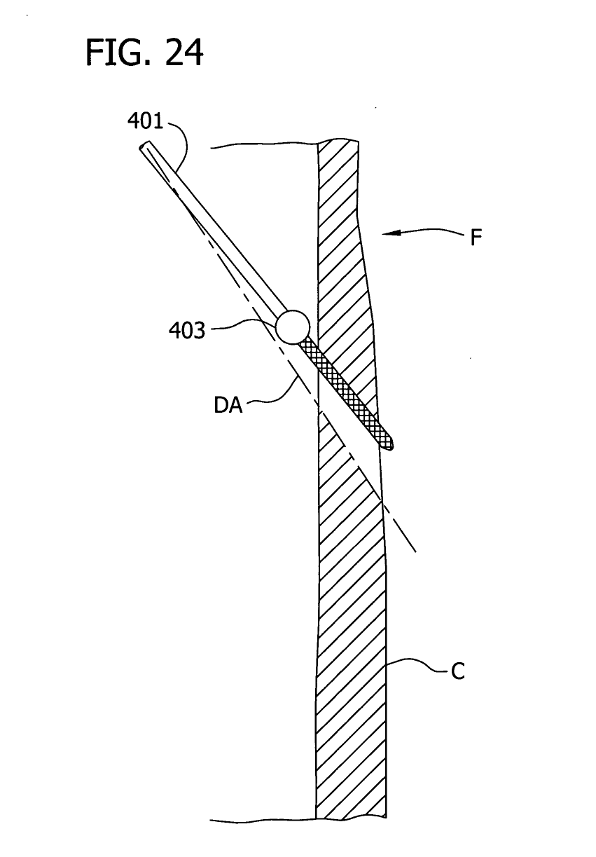

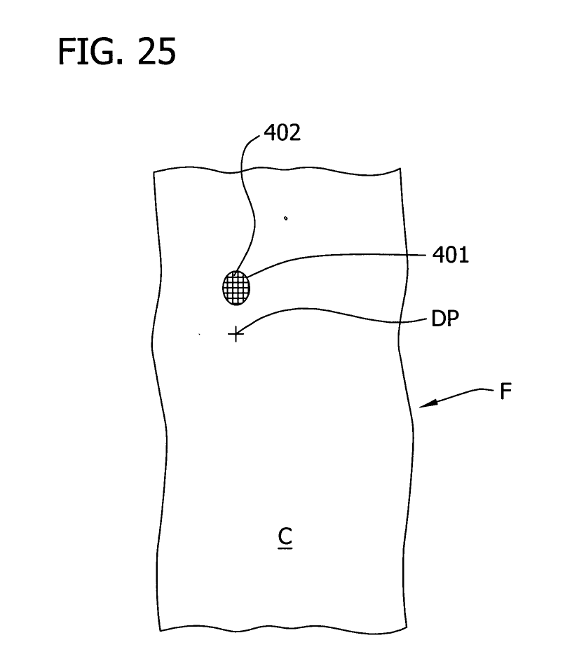

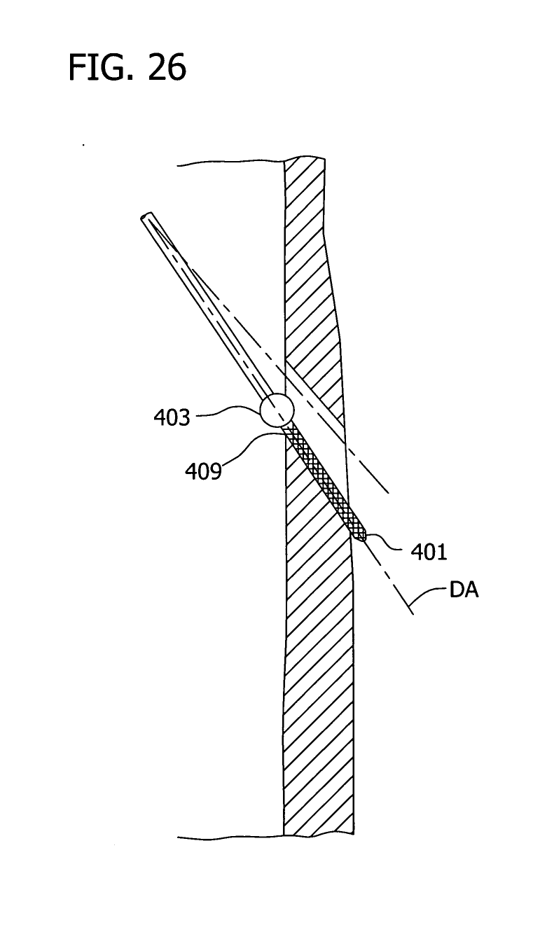

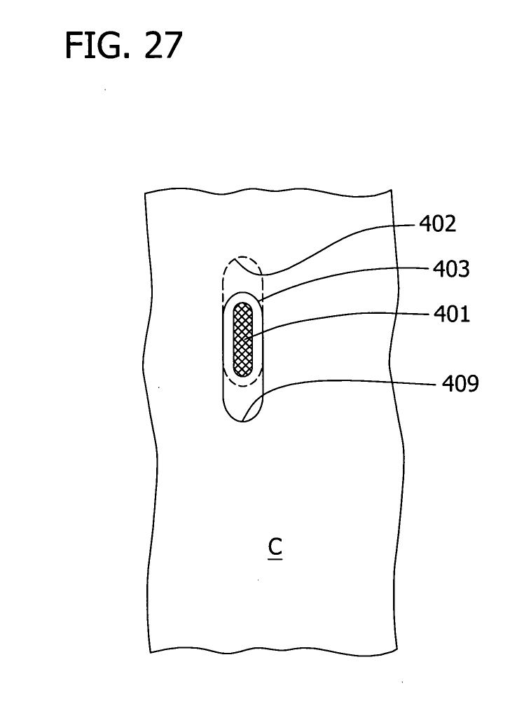

Referring to FIG. 23, to incrementally adjust the actual angle AA of the guide pin, the guide pin is first removed. (In FIGS. 23-24 dashed line is new desired guide pin angle DA, in FIG. 25 the “+” symbol is a desired exit point DP.) A side-cutting burr 401 with a ball stop 403 is inserted through the same hole 402 (FIGS. 24, 26). The side-cutting burr 401 is attached at its proximal end to a regular or high-speed drill. While the drill rotates the burr 401, the burr is slowly angled to cause the burr to go into a more vertical or horizontal inclination in relation to the femoral shaft S (FIGS. 26-27) and form a slot 409 much wider than hole 402. The burr 401 is then removed and the guide pin 405 is inserted in the slot 409. Care is taken to keep the guide pin in the correct orientation by angling it against the desired side of the slot 409. Note the actual angle AA of the guide pin 405 can be monitored directly with a goniometer (angle guide) during open surgery, fluoroscopically or via computer assistance during antegrade approach MIS, or by other suitable method. Cannulated drill-reamer 411 (or ‘dreamer’) is then passed over the guide pin (FIG. 28). The cortical bore B is formed while taking care to monitor the angle of the guide pin 405 visually, fluoroscopically, or via computer assistance.

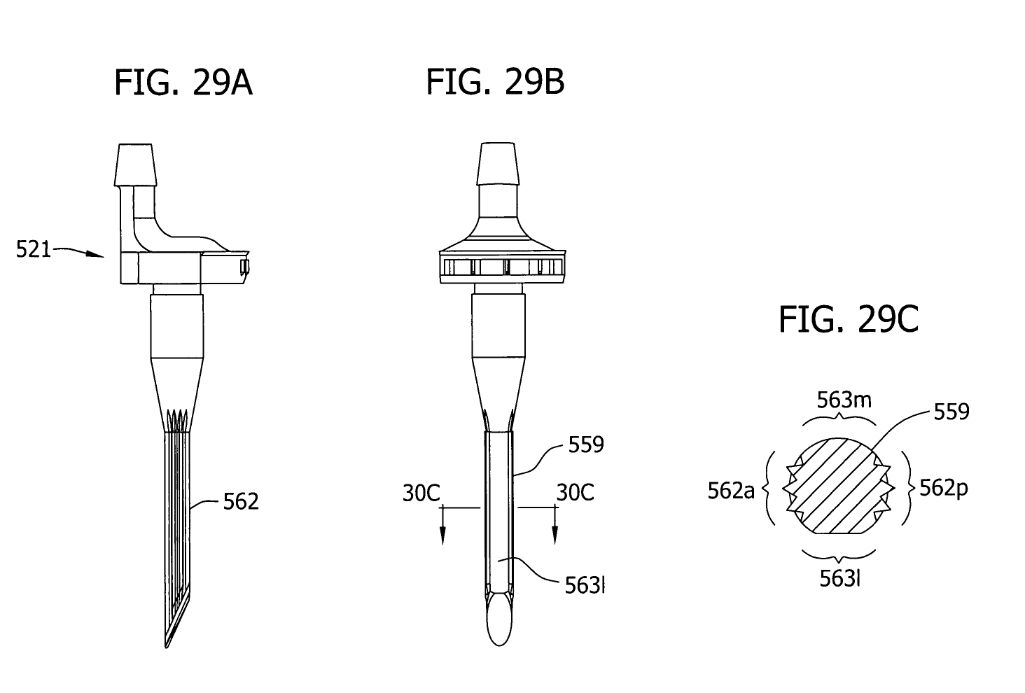

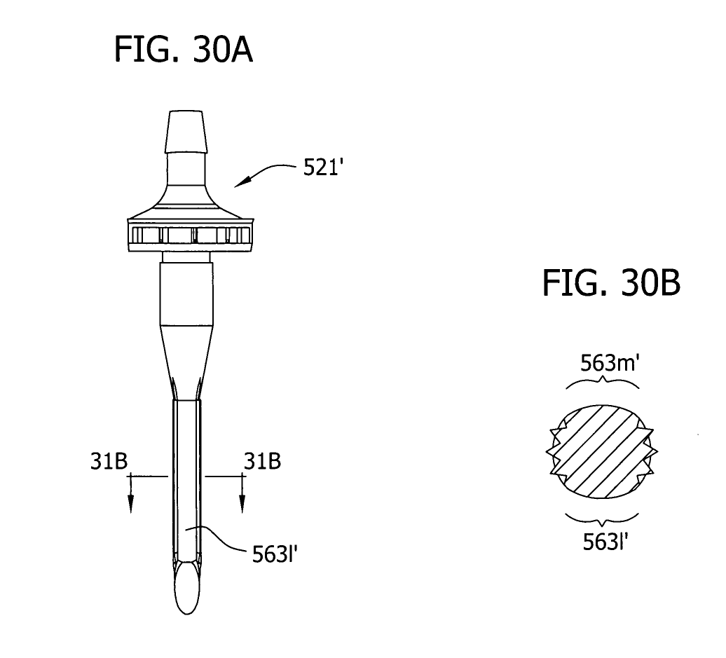

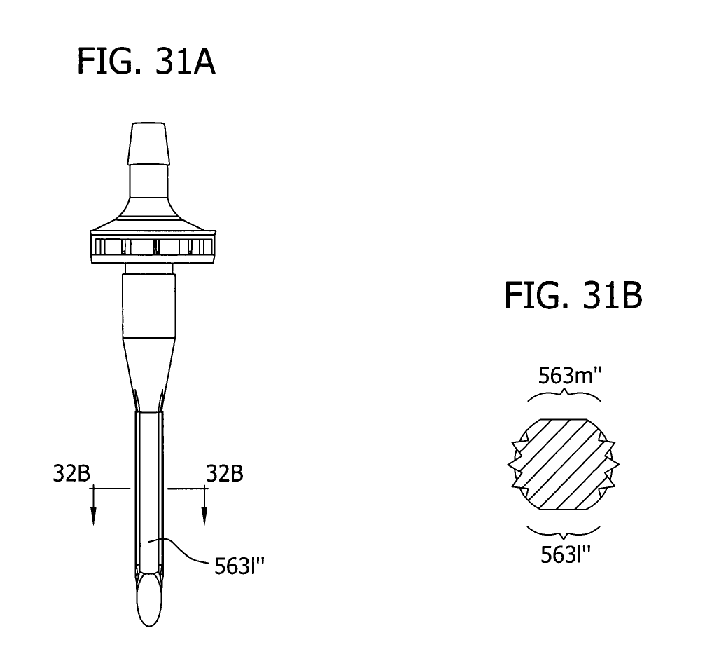

Referring to FIGS. 29A-29C, non-splined section 563l (on the lateral or superolateral side of the stem distal portion 559) is flat, rather than semi-circular (compare FIG. 29C to FIG. 7). The other non-splined section 563m and the splined sections 562a, 562p are shaped the same as in the FIG. 7 embodiment. However, in FIGS. 30A-30B, both non-splined sections 563l′ and 563m′ are modified, both being curved to give the stem an oval shape in cross-section. In FIGS. 31A-31B, the non-splined sections 563l″ and 563m″ are both flat.

All of these embodiments further decrease the risk of fracture as compared to the partially splined stem 31 of FIG. 7. Impacting the stem through the cortical bore B places the walls of the bore in tension and deforms the cortex somewhat. This deformation may slightly change the shape of the bore B to a more oval cross-section, with the long axis of the oval oriented anterior to posterior. In addition, the stem tends to deviate into varus (increased horizontal inclination). The bore deformation and the stem deviation put pressure on the superior edge of the bore exit, which is the thinnest bone. The flat on the superolateral side of the stem distal portion eliminates or inhibits contact between the stem and both the superior edge of the bore exit and the superolateral wall of the bore.

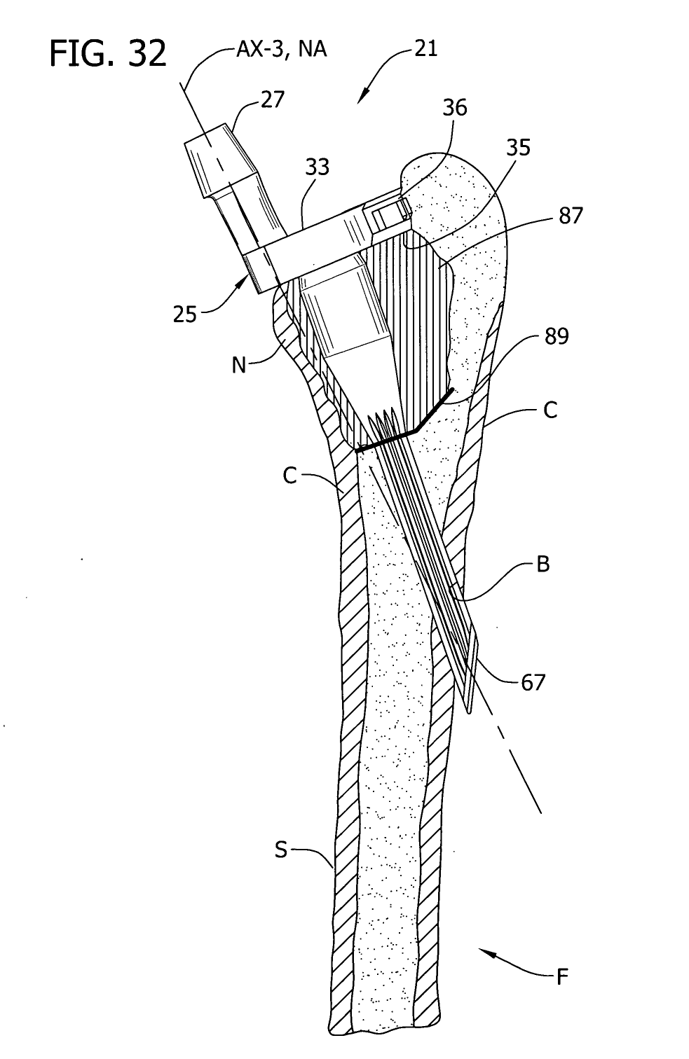

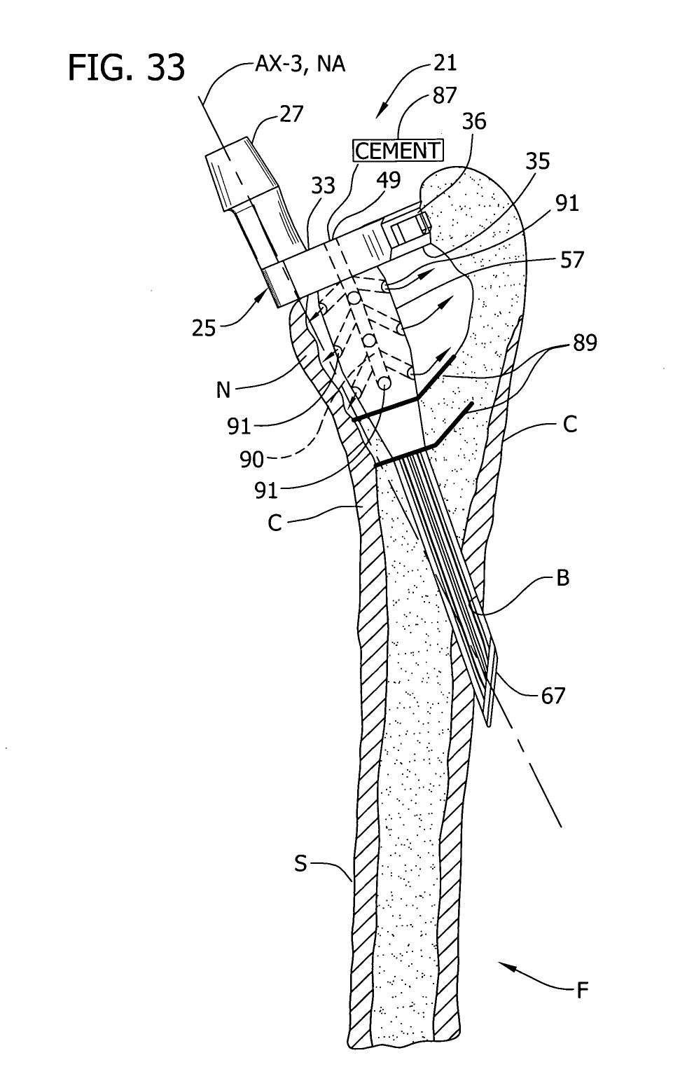

In another embodiment shown in FIGS. 32-33, the proximal portion 57 of the stem 31 is cemented to the surrounding bone. Cement 87 acts as a grout to stabilize the prosthesis 21 and limit the area of bone splinting. It is to be understood that cement is unnecessary in most applications of prosthesis 21. However, cement 87 may be advantageous in marginal cases, e.g., cases of extreme osteoporosis or in femurs that have previously received a conventional intramedullary prosthesis and suffered the resulting proximal femoral bone loss.

As shown in FIG. 32, the stem includes a restrictor 89 disposed around the stem central portion 58. The restrictor 89 is ring-shaped and is angled upwardly to catch the cement 87 and inhibit the cement from migrating downward around the distal portion 57. The restrictor 89 is suitably positioned on the stem just prior to implantation by sliding it upward from the distal tip 67. The restrictor is suitably made of polyethylene, and the cement is polymethyl methacrylate (PMMA). In this embodiment, it may be advantageous to modify the lower surface 35 of the collar 25 to include texturing, knurling or machined ribs (not shown) to promote a mechanical interlock between the collar and the cement. The shape of the restrictor 89 may also be modified to conform to the shape of the canal, e.g., to avoid the cortex.

A suitable method of implantation is similar to that described above, especially with respect to prosthesis 21, except that the stem 31 and the collar 25 are left a few centimeters short of being fully seated and cement is injected around the proximal portion 57. As the stem and collar are driven further to a fully seated position, the stem and collar act as a syringe to pressurize the cement within the surrounding bone. Thereafter, any excess or extruded cement is removed. Note that the cement does not fill the area around the entire stem, or even fill all of the area of proximal femur, but rather is limited to the area around the proximal portion 57 of the stem 31.

Referring to FIG. 33, a similar embodiment includes a passageway 90 and ports 91 in the stem proximal portion 57 that are in fluid communication with the hole 49 in the upper surface of the collar. Also, two restrictors 89 are spaced apart on the central portion 58 of the stem. In this embodiment, the stem and collar may be fully seated in the bone, and the cement 87 may thereafter be injected through the passageway (see arrows in FIG. 33). Again, the restrictors 89 inhibit downward flow of the cement. It is contemplated that the ports 91 may be oriented mostly proximally and medially because the cement will tend to bond only to the cortical bone, not to the marrow.

Testing of a prosthesis embodying aspects of the invention was performed and compared with prior art conventional prostheses. The inventive prosthesis was aligned on an intercept axis as described above. The testing showed that the prosthesis of the invention is superior to conventional prostheses in terms of restoring natural strain to the femur. The prosthesis reduced stress shielding and restored about 100% of strain (normalized or natural strain) to the proximal (upper) femur. Advantageously, the modular construction aids in consistently aligning the seat with the bore and in producing optimal strain. Relatedly, the splined configuration of the implant helps to ensure proper alignment of the stem. The lip in the collar inhibits “pistoning” of the prosthesis after implantation.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A bone prosthesis for implantation at a joint, the prosthesis comprising a stem sized and shaped for implantation in a bone at the joint, the stem having a proximal portion, a distal portion and a longitudinal axis extending therethrough, the distal portion having an outer periphery including splined sections of longitudinally extending splines and non-splined sections separating the splined sections, the splined sections and non-splined sections being constructed and arranged for facilitating implantation and for inhibiting cracking of the bone.

2. A bone prosthesis as set forth in claim 1 wherein the splined sections are disposed opposite one another on the outer periphery.

3. A bone prosthesis as set forth in claim 2 wherein the stem is cylindrical.

4. A bone prosthesis as set forth in claim 3 wherein one of the splined sections is oriented for implantation anteriorly in the femur and wherein another of the splined sections is oriented for implantation posteriorly in the femur.

5. A bone prosthesis as set forth in claim 4 wherein one of the non-splined sections is oriented for implantation medially in the femur and wherein another of the non-splined sections is oriented for implantation laterally in the femur.

6. A bone prosthesis as set forth in claim 5 wherein the non-splined sections are generally smooth.

7. A bone prosthesis as set forth in claim 6 wherein at least one of the non-splined sections is shaped to avoid contact with the bone.

8. A bone prosthesis as set forth in claim 6 wherein the lateral non-splined section is flat to avoid contact with the bone.

9. A bone prosthesis as set forth in claim 6 further comprising a distal tip at an end of the distal portion and wherein the tip includes a non-splined, curved leading edge and a smooth non-splined section adjacent the leading edge for centering of the stem during implantation.

10. A bone prosthesis as set forth in claim 9 wherein the distal tip is angled from the lateral non-splined section to the medial non-splined section.

11. A bone prosthesis as set forth in claim 6 wherein about 30% to about 95% of the outer periphery of the distal portion is generally smooth.

12. A bone prosthesis as set forth in claim 1 wherein each splined section includes at least one spline.

13. A bone prosthesis as set forth in claim 1 wherein each splined section includes at least two splines.

14. A bone prosthesis as set forth in claim 1 wherein the prosthesis further comprises a collar having a first surface and a second surface generally on an opposite side of the collar for engaging the bone and transferring load to the bone, the stem extending from the second surface, and a neck extending outwardly from the first surface and adapted to receive a ball thereon.

15. A bone prosthesis as set forth in claim 14 wherein the stem is separable from the collar.

16. A bone prosthesis as set forth in claim 15 wherein the prosthesis is adapted for transosseous implantation in a femur.

17. A bone prosthesis as set forth in claim 16 wherein one of the splined sections is oriented for implantation anteriorly in the femur and wherein another of the splined sections is oriented for implantation posteriorly in the femur.

18. A bone prosthesis for implantation at a joint having a bore formed in a bone at the joint, the bore having an entrance at one side of the bone and an exit at an opposite side, the prosthesis comprising:
a stem adapted for implantation through the bore, the stem including
a distal portion having an outer periphery including splined sections of longitudinally extending splines,
a distal tip extending from the distal portion, the tip including a smooth, curved leading edge and a non-splined, smooth section disposed between the leading edge and the distal portion for facilitating insertion of the tip through the entrance and through the exit of the bore and for facilitating centering of the distal portion, and
the distal portion and distal tip being formed integrally as one piece.

19. A bone prosthesis as set forth in claim 18 wherein the tip has an acerate shape and is angled between about 15 and about 45 degrees.

20. A femoral prosthesis for transosseous implantation in a femur having a bore and an adjacent seat formed therein, the prosthesis comprising a collar, a neck mounted on one side of the collar, and a stem extending from the collar on the opposite side of the collar from the neck, the collar including a lip for engaging the seat formed in the femur so as to inhibit withdrawal of the prosthesis from the seat and the bore while allowing compression of the prosthesis against the bone.

21. A prosthesis as set forth in claim 20 wherein the stem includes a proximal portion and wherein the lip is formed to be press fit into the seat in the femur so as to force the proximal portion of the stem medially.

22. A prosthesis as set forth in claim 21 wherein the collar includes an opening adjacent the lip for receiving bone graft material for encouraging bone ingrowth into the collar.

23. A prosthesis as set forth in claim 21 wherein the stem includes a distal portion having splines thereon, the splines being sized for an interference fit with the bore in the femur.

24. A femoral prosthesis for transosseous implantation in a femur having a bore and an adjacent seat formed therein, the prosthesis comprising a collar, a neck mounted on one side of the collar, and a stem extending from the collar on the opposite side of the collar from the neck, the collar being sized and shaped for engaging the seat formed in the femur so as to inhibit withdrawal of the prosthesis from the seat and the bore while allowing compression of the prosthesis against the bone.

25. A femoral prosthesis for transosseous implantation in a femur, the prosthesis comprising a neck adapted to receive a ball thereon and having a neck longitudinal axis, a collar on which the neck is mounted and a stem extending from the collar on the opposite side of the collar from the neck, the stem including a proximal portion adjacent the collar, a central portion and a distal portion opposite the proximal portion, the distal portion having a distal tip, the proximal and central portion being symmetric about a stem longitudinal axis, the stem longitudinal axis being angled relative to the neck longitudinal axis and forming an acute angle relative to the collar.

26. A femoral prosthesis as set forth in claim 25 wherein the angle between the neck axis and the stem axis is between about 3 and about 15 degrees.

27. A femoral prosthesis as set forth in claim 25 wherein the neck axis is angled obliquely with respect to the collar.

28. A femoral prosthesis as set forth in claim 25 wherein the stem is separable from the collar.

29. A femoral prosthesis as set forth in claim 25 wherein the distal portion, except the distal tip of the distal portion, is symmetric about the stem longitudinal axis.

30. A prosthesis adapted for transosseous implantation in a bore through a femur extending across the femoral medullary canal and through a side of the femur, the prosthesis comprising:
a first assembly including:
a collar having a first side and a second side opposite the first side adapted to engage the femur, and
a neck fixed to the first side of the collar and adapted to receive a ball thereon, and
a second assembly including:
a generally straight stem adapted for transosseous implantation in the bore,
the second assembly being securable to the first assembly for extending from the second side of the collar.

31. A prosthesis as set forth in claim 30 wherein the collar includes an opening in its second side for receiving the stem.

32. A prosthesis as set forth in claim 30 wherein the opening extends through to the first side.

33. A prosthesis as set forth in claim 30 wherein the opening includes a sleeve for receiving the stem.

34. A prosthesis as set forth in claim 30 wherein the second side of the collar includes a porous metal or bioactive coating for encouraging bone ingrowth into the prosthesis.

35. A prosthesis as set forth in claim 34 wherein the collar of the first assembly includes a heat treated coating and wherein the second assembly is not heat treated to prevent warpage of the stem.

36. A method for implanting a femoral prosthesis in a femur, the prosthesis including a stem having a stem axis, the femur having a shaft, a neck at the upper end of the shaft at the medial side of the femur and a trabecular stream, the method comprising the steps of:
determining the axis of the trabecular stream of the femur;
forming a seat on the femoral neck;
drilling a bore along a line through the shaft of the femur to extend from the neck of the femur down toward the lateral side of the femur along a line intersecting the trabecular stream generally at the lateral side of the femur so as to increase the bore length through the femur and to decrease the bending moment on the prosthesis;
inserting the stem of the prosthesis in the bore extending through the shaft to the lateral side of the femur.

37. A method as set forth in claim 36 wherein the prosthesis includes a neck having a neck longitudinal axis angled relative to a longitudinal axis of the stem, and the method further comprises installing the prosthesis so that the neck longitudinal axis is parallel or co-linear with the trabecular stream.

38. A method as set forth in claim 37 wherein the stem includes splined sections thereon, and wherein the insertion step includes orienting the stem so that a splined section is positioned anteriorly in the femur and another of the splined sections is positioned posteriorly in the femur.

39. A method as set forth in claim 38 wherein the stem includes non-splined, smooth sections, and wherein the insertion step includes orienting the stem so that one of the non-splined sections is positioned superolaterally in the femur and another of the splined sections is positioned inferomedially in the femur.

40. A method as set forth in claim 38 further comprising making a single incision prior to cutting the neck of the femur, and wherein the method requires no additional incisions.

41. A method as set forth in claim 37 wherein the drilling step includes determining the line intersecting the trabecular stream and placing a guide pin on the line by use of a computerized positioning system.

42. A method as set forth in claim 36 wherein the drilling step includes using a fenestrated guide to cool the bone during drilling.

43. A method as set forth in claim 36 further comprising inserting cement around the stem.

44. A method for implanting a femoral prosthesis having a stem in a femur, the femur having a shaft, a neck at the upper end of the shaft at the medial side of the femur and a trabecular stream, the method comprising the steps of:
determining the axis of the trabecular stream of the femur;
forming a seat on the femoral neck;
drilling a bore along a line through the shaft of the femur co-linear with the medial trabecular stream generally at the lateral side of the femur so as to increase the bore length through the femur and to decrease the bending moment on the prosthesis;
inserting the stem of the prosthesis in the bore extending through the shaft to the lateral side of the femur so that a stem axis is co-linear with the medial trabecular stream.

45. A method as set forth in claim 44 wherein the stem includes splined sections thereon, and wherein the insertion step includes orienting the stem so that a splined section is positioned anteriorly in the femur and another of the splined sections is positioned posteriorly in the femur.

46. A method for implanting a femoral head-neck prosthesis in a femur, the prosthesis including a stem having a stem axis, and a periphery including splined sections and non-splined, smooth sections, the femur having a shaft, a neck at the upper end of the shaft at the medial side of the femur and a trabecular stream, the method comprising the steps of:
forming a seat on the femoral neck;
drilling a bore through the shaft of the femur to extend from the neck of the femur down through the lateral side of the femur;
inserting the stem of the prosthesis in the bore and orienting the stem so that one of the non-splined sections is positioned superolaterally in the femur and another of the non-splined sections is positioned inferomedially in the femur.

47. A prosthesis adapted for transosseous implantation in a bore through a femur extending across the femoral medullary canal and through a side of the femur, the prosthesis comprising:
a first assembly including:
a collar having a first side and a second side opposite the first side adapted to engage the femur,
a neck mounted on a first side of the collar and adapted to receive a ball thereon, and
a proximal stem secured to the second side of the collar, and
a second assembly including:
a distal stem adapted for implantation in the bore,
the second assembly being securable to the first assembly for extending from the proximal stem.

48. A prosthesis as set forth in claim 47 wherein the neck has an axis and the distal stem has an axis co-linear with the neck axis.

49. A bone prosthesis for implantation in a bore formed in a bone at a joint, the prosthesis comprising:
a collar having a first side and a second side opposite the first side adapted to engage the femur,
a neck mounted on a first side of the collar and having a longitudinal neck axis,
a stem sized and shaped for implantation in a bore through the bone, the stem including a proximal portion and a distal portion having a longitudinal distal axis generally co-linear with the neck axis and offset from the proximal portion.

50. A bone prosthesis as set forth in claim 49 wherein the proximal portion of the stem includes a curved section adapted to preserve cortical bone in a femur.

51. A bone prosthesis as set forth in claim 50 wherein the proximal portion includes a medial side, the medial side being curved to preserve cortical bone.

52. A method for implanting a femoral prosthesis in a femur, the prosthesis comprising a collar and stem including a proximal portion, a distal portion and a cement restrictor around the proximal portion, the femur having a shaft, a neck at the upper end of the shaft at the medial side of the femur, the method comprising the steps of:
forming a seat on the femoral neck;
drilling a bore along a line through the shaft of the femur to extend from the neck of the femur down through the lateral side of the femur;
partially inserting the stem of the prosthesis in the bore extending through the shaft to the lateral side of the femur;
placing cement around the proximal portion of the stem such that the restrictor inhibits the cement from flowing down toward the distal portion; and
impacting the prosthesis into the bone so that the collar contacts the seat.

53. A method as set forth in claim 48 wherein the placing step includes introducing the cement through channels formed in the proximal portion of the stem.

54. A method of incrementally adjusting a location of a guide pin in forming a bore for transosseous prosthetic implantation, the method comprising
withdrawing the guide pin from a first guide hole;
determining a location of a final guide slot;
placing a side-cutting burr in the first guide hole; and
rotating the burr while forcing the burr against one edge of the hole to expand the hole and thereby form the final guide slot
inserting the guide pin in the guide slot.

55. A guide for aligning a shaft having a cutting surface thereon during the implantation of a prosthesis, the guide comprising:
a cylindrical body having a top, a bottom, an inner wall and an outer wall, said inner wall defining an opening for allowing the shaft to pass therethrough;
at least one passage extending from the top of the body to said bottom of the body for allowing fluid to pass through said guide for direct fluid contact with said cutting surface.

56. A guide as set forth in claim 55 wherein said passage is positioned between the outer and inner walls of the body.

57. A guide as set forth in claim 56 wherein the bottom of the body has a generally frustoconical shape.

58. A guide as set forth in claim 56 wherein said guide comprises at least one inlet passage for allowing fluid to pass from the top of the body to the bottom of the body, and at least one outlet passage for allowing fluid to pass from the bottom of the body to the top of the body.

59. A guide as set forth in claim 58 wherein said guide comprises a plurality of inlet passages and a plurality of outlet passages.

60. A guide as set forth in claim 56 wherein said passage comprises a single aperture located on the top of the body for receiving fluid into the body, and a plurality of apertures located on the bottom of the body for allowing the fluid to exit the body.

61. A guide as set forth in claim 55 in combination with a drill having a shaft with a reamer mounted thereon.

62. A guide as set forth in claim 55 in combination with at least one catheter.

63. A femoral prosthesis adapted for transosseous implatation comprising a neck, a collar, and a stem, the stem comprising a first diameter cylinder tapering to a smaller second diameter in a distal portion of the stem, wherein a medial side of the stem lies along a straight line.
